(19) United States
(12) Patent Application Publication
(10) Pub. No.: US 2020/0324099 A1
(43) Pub. Date: Oct. 15, 2020

(54) ACOUSTIC WAVE MEDIATED NON-INVASIVE DRUG DELIVERY

(71) Applicant: Mupharma Pty Ltd, Toorak, Victoria (AU)

(72) Inventors: Harry Unger, Toorak, Victoria (AU); Mark Unger, Toorak, Victoria (AU)

(21) Appl. No.: 16/090,761

(22) PCT Filed: Apr. 6, 2017
(86) PCT No.: PCT/AU2017/000083

§ 371 Date: Oct. 2, 2018

(30) Foreign Application Priority Data
Apr. 6, 2016 (AU) 2016901280

(51) Int. Cl. A61M 37/00; A61F 9/00; A61N 5/06; A61K 31/675; A61P 27/02; A61K 9/00; A61K 41/00

(52) U.S. Cl. CPC A61M 37/0092; A61F 9/0017; A61N 5/062; A61K 31/675; A61P 27/02; A61K 9/0048; A61M 2037/0007; A61K 9/0009; A61M 2205/0294; A61M 2210/0612; A61N 2005/0661; A61N 2005/0643; A61K 41/0047

(57) ABSTRACT

The present invention relates to a device, comprising: an agent carrier comprising an agent transfer surface for delivery of an agent into a tissue, wherein the agent carrier comprises or is acoustically couplable to a piezoelectric substrate; an electrode electrically couplable to the piezoelectric substrate; and a controller electrically couplable to the electrode and configured to apply an electrical signal to the electrode to propagate an acoustic wave on and/or in the piezoelectric substrate which is capable of delivering the agent from the device into the tissue. Methods of using the device for non-invasive delivery of agents into target tissues are also disclosed.

### INCORPORATION BY CROSS-REFERENCE

The present application claims priority from Australian provisional patent application number 2016901280 filed on 6 Apr. 2016, the entire contents of which are incorporated herein by cross-reference.

### TECHNICAL FIELD

The present invention relates to acoustic wave mediated non-invasive drug delivery.

### BACKGROUND

### Delivery of Drugs to the Body

For over 100 years, the hypodermic needle and syringe has remained as the standard method of delivering drugs to the body when oral, rectal or topical medication is impractical or cannot be used. To overcome problems associated with oral or rectal drug administration, including poor absorption of certain drugs from the gastrointestinal tract, unwanted gastro-intestinal side effects of drugs and the degradation of drugs in the gut and the liver, intramuscular, intravenous or subcutaneous injections are used. Skin is a formidable barrier and whilst topical application of drugs to skin has been used, poor absorption of the majority of drugs in particular biologics and molecules larger than 500 Dalton, as well as the development of local allergic reactions have limited this route for drug delivery to a small group of drugs.

Systemic delivery of drugs to the body is desirable when a drug is intended to affect biological systems or illnesses that affect many parts of the body. It is not desirable when a drug is intended to only affect certain tissues or organs, which is more often the case. Independent of the route of delivery, the systemic effect of drugs commonly includes undesirable side effects in one or more tissues or organs. If only one organ is affected, such as the eye, drug delivery directly to it is highly desirable as the drug has its therapeutic effect on the target tissues where the disease or condition manifests, reduces the dose required to achieve the desired therapeutic effect and minimises unwanted side effects in other organs and biological systems.

A non-invasive drug delivery device (“NIDDD”) capable of safely, efficiently, effectively and practically delivering a wide range of drugs to various tissues of the body would address many unmet medical needs, some of which are discussed below.

### Ophthalmology

An unmet medical need in the field of ophthalmology is to replace intraocular injections as the conventional means of delivering biologic drugs and certain other drugs to the retina and choroid at the back of the eye. As an example, for the treatment of Wet Age Related Macular Degeneration, a potentially blinding eye condition, patients currently require long term, usually monthly, intra-ocular injections of particular immunoglobulins. Apart from the associated patient fear and discomfort, the injection itself carries significant risk from physical trauma to the ocular contents and infection. A NIDDD capable of delivering biologic drugs and other drug classes to one or more coats of the eye without intra-ocular injection would be highly advantageous as it would minimise or overcome many existing risks and problems associated with intra-ocular injections.

### Immunology

In the field of immunology, there is (among other things) an unmet medical need to induce mucosal immunity in the body in a safe and effective manner. Also, there is an unmet medical need to eliminate or reduce the dependence on continuous refrigeration (the “cold chain”) for the transportation of many vaccines to the end user, particularly for use in third world vaccination programs.

Whilst the body has an innate immune system to defend against many pathogens, the role of vaccination is to create adaptive immunity which broadly speaking has two types of immune mechanisms, systemic and mucosal. Vaccination historically is provided by injection and this, by delivering antigens to subcutaneous or muscle tissue, predominantly creates systemic immunity. Delivery of vaccines primarily to specialised cells residing in mucous membranes without breaching the epithelial layers of tissue (which is not possible with injections and other invasive drug delivery technologies), can generate immune responses, importantly, favouring the creation of strong mucosal immunity. The induction of mucosal immunity is highly advantageous for creating immunity to diseases that enter the body via mucosal tissue like influenza, HIV-AIDS, tuberculosis as well as a host of other diseases.

Vaccines administered by intramuscular, subcutaneous and intradermal injections usually fail to create high levels of protective mucosal immunity. This is because the vaccine antigen delivered by injection is primarily exposed to cells in the blood stream and deeper tissue spaces which provide the mechanism for systemic rather than mucosal immunity. A NIDDD that is capable of delivering a dose of a vaccine within mucosal tissues that is sufficient to create a mucosal immune response would address an important unmet medical need.

Further and importantly, non-invasive delivery of drugs to specific target groups of cells in the body offers highly desirable unique treatment options not possible with injections and other invasive drug delivery technologies. Delivering significant amount of drugs to mucous membranes and eliciting mucosal immune responses is not only important in creating mucosal immunity, but it also has a potential important role to play, using immune mechanisms, in treating allergic conditions such as asthma and for the treatment of certain cancers.

Mucosal immunity, where specialised defensive cells and antibodies are located primarily within the mucous membrane, is desirable so that pathogens that gain entry to the body via mucous membranes like the respiratory, gastro-intestinal and genito-urinary mucosae are challenged at their point of entry. Injection of vaccines beneath the skin or into muscle does not create mucosal immunity of the order of that achieved by the induction of mucosal immunity at site of mucosal tissue, but instead, tends to create an immune response favouring systemic immunity where the defensive cells and antibodies circulate primarily in the bloodstream. The characteristics of the defensive cells and antibody types differ between the mucosal and systemic immune systems.

Mucosal surfaces are a major portal of entry for many pathogens that cause infectious diseases worldwide. Vaccines capable of eliciting mucosal immune responses can fortify defenses at mucosal front lines and protect against infection. Immunization via mucosal routes is more effective at inducing protective immunity against mucosal pathogens at their sites of entry. Recent advances in the understanding of mucosal immunity and identification of correlates of protective immunity against specific mucosal pathogens have renewed interest in the development of mucosal vaccines. Efforts have focused on efficient delivery of vaccine antigens to mucosal sites that facilitate uptake by local antigen-presenting cells to generate protective mucosal immune responses. The induction of strong mucosal immunity is important for the development of effective vaccines against infections such as, for example, Influenza, Tuberculosis and HIV. Mucosal vaccines offer several advantages over parenteral immunization. For example, (i) ease of administration; (ii) non-invasiveness; (iii) high-patient compliance and (iv) suitability for mass vaccination.

### Occupational Health and Safety

With the increased awareness about accidental needle-stick injuries and the consequential risk of exposure to Hepatitis C and HIV-AIDS, coupled with an escalating emphasis on occupational health and safety, needle-free drug delivery by NIDDD technologies provide further benefits.

### Current Alternatives to Oral and Conventional Injection Drug Delivery Technologies

The conventional methods for injecting involve gaining access to subcutaneous, intradermal, intramuscular and intravenous regions and are therefore invasive. In an effort to overcome the need for conventional injections, a number of technologies using a variety of methods and routes of administration have been developed. The principal aim of these technologies has been to deliver drugs “needle-free” meaning without a classical hypodermic needle and syringe rather than “non-invasively” as defined above.

Micro-needle drug delivery involves the creation of multiple micro-punctures through one or more layers of skin, to deliver drugs into the body. The micro-needles are commonly housed in a patch that is applied to skin for a variable period of time. In comparison to conventional injections, this technique is minimally invasive but it is not “non-invasive.”

Fluidic “jet” injectors are “needle-free” but they do disrupt the surface tissue to deliver drugs to the subcutaneous tissue.

Iontophoresis is a method whereby a drug becomes an integral component of an electric current that is established between the drug delivery device and the patient. This involves placing an electrode on the patient, usually on skin, and applying a voltage between the patient and the drug delivery device. Small, usually low molecular weight, electrically charged drugs have been delivered by this technique and dependent on the drug and the desired therapeutic effect, the drug is delivered over a significant period of time commonly, minutes to hours. Large uncharged molecules like many biologics are unsuitable for delivery. The principal application has been to deliver suitable drugs through skin.

Electroporation can also be used to deliver drugs into cells by applying intense, high-voltage electric pulses of short duration repeatedly to transiently permeabilise cell membranes by creating pores within them and thereby allowing the transport of molecules that would not normally be transported through intact cellular membranes. The principal application has been to deliver suitable drugs through skin.

Aerosols of drugs, either dissolved in liquid or in powder form, is non-invasive and has been used to deliver drugs via the intra-nasal or pulmonary (lung) routes for both local and systemic effect. Examples are decongestant nasal sprays and pulmonary nebulisers for the treatment of asthma.

The intranasal and pulmonary routes have both been researched in an effort to create mucosal immunity. Problems with these routes include: concern with the delivery of antigens directly to the brain via the olfactory nerves at the roof of the nose; variable absorption from both nose and lung mucosa as a result of variations in the dose actually reaching the tissues and inactivation of the vaccines though the variable hydration of the tissues and by resident mucous and enzyme protection. Predictable delivery of drugs by these routes may require patients to learn how to inhale the nebulised material properly. This is dependent on the device, whether drug is delivered directly to the nose or lung airway as a bolus or is delivered into a receptacle that allows for rebreathing. The treatment of asthma serves as an example where bolus puffer use may be unsuccessful in delivering drugs to the lung via the mouth. A variety of masks and rebreathing chambers have been developed to overcome this problem and improve compliance.

Sonophoresis is also a non-invasive technique that utilises ultrasound in order to make tissues briefly more permeable to the entry and transport of drugs through them. It is typical for drugs aimed at achieving a systemic therapeutic outcome to seek deeper penetration to facilitate systemic drug delivery via blood vessels that lie below the tissue surface. Sonophoresis can be used for both local and systemic drug delivery.

### Surface Acoustic Waves Used in Non-Invasive Drug Delivery Technologies

A surface acoustic wave (SAW) is an acoustic wave traveling along the surface of a material as a result of the elasticity of the material and its piezoelectricity i.e. deformation due to electric stimulation and vice versa, with amplitudes that typically decay exponentially with depth into the substrate that generates them. Electronic devices employing SAWs normally use one or more interdigital transducers (IDTs) to convert acoustic waves to electrical signals and vice versa by exploiting the piezoelectric properties of certain materials including quartz, lithium niobate, lithium tantalate, lead zirconate titanate and lanthanum gallium silicate. SAW devices are used as electronic components to provide a number of different functions. SAW devices have applications in radio and television, seismology and also in devices that drive microfluidic actuation for a variety of processes.

### SUMMARY

The present invention provides devices that utilises SAWs as a means of facilitating the non-invasive delivery of agents or through a target tissue. Existing devices typically utilise iontophoresis to permeate tissue surfaces which have shown limited efficacy and have demonstrated limited market penetration. The few devices which have incorporated a sonophoretic component typically use only low frequency (Hz—kHz) ultrasound as higher frequency ultrasound (e.g. MHz range, 1 MHz and above) is considered to be unsuitable for drug delivery.

The devices of the present invention can employ higher frequency ultrasound (e.g. MHz range, 1 MHz and above) to deliver agents into or through target tissues. Unlike ultrasonic transducers in which the vibration permeates through the entire bulk of the transducer, SAWs are localized on the surface of the material within a depth region of thickness in the order of its wavelength. The devices of the present invention can incorporate transducers that generate SAWs localized on the surface of the material and are hence more power efficient in imparting ultrasonic power to a liquid than transducers which generate acoustic waves throughout the entire bulk of the transducer. This power efficiency is advantageous for, among other things, decreasing the power requirements for operating a portable device that uses ultrasonic transducers to impart ultrasonic power to a liquid.

The devices of the present invention may rely entirely on SAWs to deliver agents into and in some embodiments through target tissue, and have no requirement to utilise repulsive electromotive forces or differences in electric potential to deliver the agent (e.g. iontophoresis (ionization), ionophoresis, electrophoresis, microelectrophoresis, electroosmosis, cataphoresis, electroendosmosis, electrorepulsion and the like).

The devices can be used in methods for the delivery of agents into or through target tissues for the prevention and/or treatment of conditions/diseases and/or for any other purpose. For example, as described herein the devices may be used for the delivery of agents into or through various tissues including the eye, skin and mucosal surfaces. In some embodiments, the devices may be used to deliver agents into or through mucosal surfaces and thereby induce mucosal immunity.

The common aim of non-invasive drug delivery devices to date has been to replace the need for conventional injections for systemic drug delivery. In order to achieve this, a drug must be delivered deeply enough for it to be eventually circulated via the bloodstream.

Embodiments of the present invention, aim among other things, to control the depth of delivery of a drug into tissue. Depending on the desired therapeutic effect of a particular drug, it may be beneficial that the amount of systemic delivery of a drug is limited. Some examples where limiting the range of depth of delivery of a drug into tissue is useful are the superficial mucosal delivery of vaccines to induce strong mucosal immunity and, in the case of the eye, the delivery of Riboflavin-5-phosphate-sodium to the superficial half thickness of the cornea to enable treatment of keratoconus (conical cornea) by Corneal Collagen Cross-Linking using ultraviolet light.

There is current research into the potential treatment of allergic conditions such as asthma and some cancers using immune mechanisms that are closely aligned with those encountered with the creation of mucosal immunity to vaccine antigens.

Embodiments of the present invention, by utilising a non-invasive drug delivery system that can control the depth of penetration of drugs into tissues, provide a novel approach and solution to a range of unmet medical needs.

For example, some embodiments of the present invention utilise SAWs for non-invasive delivery of agents into tissue. SAWs travel along the surface of the solid from which they are generated and are effectively confined to that surface. Using SAW, agents are expelled from the surface of the device in contact with tissue at a high velocity and, for example, at least into epithelial tissue and in some embodiments into epithelial tissue and adjacent subepithelial tissue.

In one embodiment, the present invention provides a device, comprising:

an agent carrier comprising an agent transfer surface for delivery of an agent into a tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the device into the tissue.

The piezoelectric substrate of the device may comprise a single crystal piezoelectric material, a thin-film piezoelectric material, or a combination thereof. The piezoelectric substrate may comprise any one or more of lithium niobate, tourmaline, single-crystal quartz, and/or lead zirconate titanate.

The electrical signal applied by the controller may generate a primary acoustic excitation frequency on and/or in the piezoelectric substrate in a range of 1 MHz to 10 GHz. The primary acoustic excitation frequency may correspond to the resonant frequency of the piezoelectric substrate.

The electrical signal applied by the controller may generate a primary acoustic excitation frequency on and/or in the piezoelectric substrate in a range of 1 MHz to 100 GHz of any wave type. For example, the primary acoustic excitation frequency may be more than 10⁶ Hz, more than 10⁷ Hz, more than 10⁸ Hz, more than 10⁹ Hz, more than 10¹⁰ Hz, or more than 10¹¹ Hz. The primary acoustic excitation frequency may be, for example, between 10⁶ Hz and 10⁷ Hz, between 10⁶ Hz and 10⁸ Hz, between 10⁶ Hz and 10⁹ Hz, between 10⁶ Hz and 10¹⁰ Hz, between 10⁷ Hz and 10⁸ Hz, between 10⁷ Hz and 10⁹ Hz, between 10⁷ Hz and 10¹⁰ Hz, between 10⁸ Hz and 10⁹ Hz, between 10⁸ Hz and 10¹⁰ Hz, or between 10⁹ Hz and 10¹⁰ Hz. The primary acoustic excitation frequency may correspond to the resonant frequency of the piezoelectric substrate and/or the spatial arrangement of excitation transducers electrodes.

The device (e.g. the device controller) may further comprise an acoustic generator capable of generating one or more secondary acoustic excitation frequencies of any wave type (including square, sine sawtooth) or combination thereof capable of modulating the primary acoustic excitation on and/or in the piezoelectric substrate. The secondary acoustic excitation frequency may be less than or equal to the primary acoustic excitation frequency. For example, secondary acoustic excitation frequency acoustic excitation frequency may be 1 Hz to 100 kHz, 1 Hz, less than 10 Hz, less than 10² Hz, less than 10³ Hz, less than 10⁴ Hz, less than 10⁵ Hz, less than 10⁶ Hz, less than 10⁷ Hz, less than 10⁸ Hz, less than 10⁹ Hz, less than 10¹⁰ Hz, or less than 10¹¹ Hz. The supplementary, alternative or otherwise additional acoustic frequency may, for example, be between 1 Hz and 10 Hz, between 1 Hz and 10² Hz, between 1 Hz and 10³ Hz, between 1 Hz and 10⁴ Hz, between 1 Hz and 10⁵ Hz, between 1 Hz and 10⁶ Hz, between 10 Hz and 10² Hz, between 10 Hz and 10³ Hz, between 10 Hz and 10⁴ Hz, between 10 Hz and 10⁵ Hz, between 10 Hz and 10⁶ Hz, between 10³ Hz and 10⁴ Hz, between 10³ Hz and 10⁵ Hz, between 10³ Hz and 10⁶ Hz, between 10⁴ Hz and 10⁵ Hz, between 10⁴ Hz and 10⁶ Hz, between 10⁵ Hz and 10⁶ Hz, between 10⁶ Hz and 10⁷ Hz, between 10⁶ Hz and 10⁸ Hz, between 10⁶ Hz and 10⁹ Hz, between 10⁶ Hz and 10¹⁰ Hz, between 10⁷ Hz and 10⁸ Hz, between 10⁷ Hz and 10⁹ Hz, between 10⁷ Hz and 10¹⁰ Hz, between 10⁸ Hz and 10⁹ Hz, between 10⁸ Hz and 10¹⁰ Hz, or between 10⁹ Hz and 10¹⁰ Hz. The wave type, frequency level, number and duration of additional frequencies may vary throughout the duration in which the primary acoustic excitation signal is applied to tissue. When applied to tissue the acoustic frequency signal may make it more permeable.

The acoustic wave propagated on and/or in the piezoelectric substrate may not be a bulk (lamb) wave. The acoustic wave may be a surface acoustic wave. The acoustic wave may be a Rayleigh surface acoustic wave.

The device may be incapable of utilising repulsive electromotive force to transport a charged agent into and/or through a tissue in contact with the agent transfer surface of the device.

Additionally or alternatively, the device may be incapable of generating or maintaining a difference in electric potential between the agent transfer surface of the device and the tissue surface in contact with it to consequently induce transport of the agent from the device into the tissue.

Additionally or alternatively, the device may be incapable of:

(i) utilising repulsive electromotive force to transport a charged agent into and/or through the tissue in contact with the agent transfer surface; and/or

(ii) permeating the tissue by any of iontophoresis (ionization), ionophoresis, electrophoresis, microelectrophoresis, electroosmosis, cataphoresis, electroendosmosis, and electrorepulsion.

The device may further comprise the agent.

The device may include the following features:

the agent carrier may comprise the piezoelectric substrate,

the piezoelectric substrate may comprise the agent transfer surface, and

the agent may be present on the agent transfer surface.

The device may include the following features:

the agent transfer surface may be functionalised, and/or

the agent may be lyophilised on the agent transfer surface,

to thereby retain the agent on the agent transfer surface.

The agent carrier of the device may comprise any one or more of: an absorbent material, an adsorbent material, a micro channel, a reservoir, or a combination thereof.

The agent carrier of the device may have volumteric retention capabilities and comprise any one or more of: a porous absorbent material, a porous adsorbent material, a non-porous solid material which has been micro-machined such that micro channels, reservoirs, or a combination thereof are created. The fluid contained in porous agent carriers is in contact with itself so that there is a continuous fluid medium. The fluid contained in the non-porous micro-machined material may be a continuous fluid medium.

The agent carrier of the device may comprise a network and/or multiplicity of micro channels and/or pores extending at least partially or wholly through the agent carrier to the agent transfer surface enabling retention (e.g. volumetric retention) of the agent and/or transportation of the agent to the tissue.

The agent carrier of the device may comprise a stack of layers, and the stack of layers may comprise:

a first layer comprising the agent transfer surface; and

at least one other layer,

wherein holes formed in one layer of the plurality of layers are aligned with holes in an adjacent layer and in an arrangement facilitating a plurality of holes in a plurality of layers to cooperate to form the micro channels.

The micro channels of the device may extend from the interior of the agent carrier body and terminate as pores at the agent transfer surface.

The agent transfer surface of the device may comprise a plurality of hollow micro protrusions in fluid communication with the micro channels.

The device may be further defined by the following features:

(i) the micro protrusions may not be microneedles and may not function as microneedles; and

(ii) the tissue may be intact tissue and the micro protrusions may be shaped, arranged, and/or of a length that inhibits or prevents mechanical penetration of the surface of the tissue when the agent transfer surface is in contact with the tissue during standard use of the device. “Standard use” of the device will be understood to comprise not applying the device with such force that the tissue in contact with the agent transfer surface or surrounding tissue is penetrated, pierced, or bruised.

The tissue of the embodiment above may be skin. The tissue of the above embodiment may be mucosal tissue and the micro protrusions may be shaped, arranged, and/or of a length that inhibits or prevents mechanical penetration of an intact epithelial layer of the mucosal tissue during standard use of the device.

The agent transfer surface of the device may comprise a plurality of protrusions in fluid communication with the agent reservoirs. The plurality of protrusions may extend outward from an inside of one or more of the voids and terminate at the agent transfer surface. One or more of the voids may be formed by a peripheral structure, and:

(i) the peripheral structure may terminate in a common plane with the plurality of protrusions; or

(ii) the plurality of protrusions may extend outward from the void beyond the peripheral structure; or

(iii) the plurality of protrusions may terminate in a plane and the peripheral structure may terminate short of the plane such that the plurality of protrusions extend beyond the peripheral structure.

The device may be further defined by the following features:

(i) the plurality of protrusions in fluid communication with the agent reservoirs may not be microneedles and may not function as microneedles; and/or

(ii) the tissue may be intact tissue and the plurality of protrusions in fluid communication with the agent reservoirs may be shaped, arranged, and/or of a length that inhibits or prevents mechanical penetration of the surface of the tissue when the agent transfer surface is in contact with the tissue during standard use of the device. “Standard use” of the device will be understood to comprise not applying the device with such force that the tissue in contact with the agent transfer surface or surrounding tissue is penetrated, pierced, or bruised.

The protrusions and micro protrusions in devices of the present invention may not have a needle-like tip, that is, they do not narrow to a point such that their width does not decrease to near zero at the tip. The cross-section of the protrusions/micro protrusions may be relatively constant, at least near their tip, and most preferably along their whole length. In most cases the width of the protrusions/micro protrusions may not narrow by more than 20%, and preferably less than 10% towards its tip.

The protrusions/micro protrusions may have a tip diameter greater than 10 μm. The protrusions/micro protrusions may have a tip with a characteristic lateral dimension of more than 1 mm, more than 2 mm, more than 3 mm, more than 4 mm or more than 5 mm. Thus the scale of the protrusions/micro protrusions also generally differs generally from that of microneedles. The protrusions/micro protrusions do not enter an intact epithelial surface of the target tissue during standard use of the device. The protrusions/micro protrusions may aid in stabilizing the device by the frictional force they apply when the device is placed in contact with the tissue. This can be particularly advantageous on mucous membranes that tend to have a low friction surface due to local mucous secretions. The protrusions/micro protrusions may generally have a height to width aspect ratio (across their characteristic lateral dimension) of between 1:1 to 10:1. Whilst higher aspect ratios may be used if it is difficult to achieve acceptable strength so that the protrusions/micro protrusions can withstand handling, loading and/or application of ultrasonic energy without damage.

The cross-sectional shape of the protrusions may significantly alter their strength and thus may be chosen accordingly.

The protrusions may occupy more than 5% of the volume surrounding them in which agent is carried. It is preferable that this percentage be high enough so that the capillary force or other forces retain the agent within the agent carrier body against gravity or other forces caused by normal handling.

When used with water-like agents, the protrusions may have a density of projections of greater than 5% and most preferably greater than 10% of the total area collectively occupied by the protrusions. It should also be appreciated that when using more viscous agents, (e.g. protein rich agents) the density of protrusions, or their size and/or wall surface area, can be lowered.

The tissue of the embodiment above may be skin. The tissue of the above embodiment may be mucosal tissue and the plurality of protrusions may be shaped, arranged, and/or of a length that inhibits or prevents mechanical penetration of an intact epithelial layer of the mucosal tissue during standard use of the device.

The agent carrier of the device may be formed from any one or more of a polymer, a metal, silicon, a ceramic, and/or plastic.

The agent of the device may comprise a solid or a combination of a solid and a liquid. The agent may comprise a solid comprising powder, granules, or a combination thereof. The agent may be lyophilised. The agent may comprise a therapeutic agent, a prophylactic agent, a diagnostic agent, a cosmetic agent, or any combination thereof. The agent may be selected from the group consisting of: a protein, a peptide, a polypeptide, an immunogenic agent, a vaccine, a biomimetic, a biosimilar, a biomaterial, a macromolecule, a small molecule, a sugar, a nucleic acid, an antibody, a drug, a nanoparticle, and any combination thereof.

In another embodiment, the present invention provides a method for delivering an agent to an internal layer within a target tissue, the method comprising:

contacting the target tissue with the agent transfer surface of a device of the present invention, and

applying an electrical signal to the electrode of the device to propagate an acoustic wave on and/or in the piezoelectric substrate of the device, and thereby deliver the agent through the agent transfer surface to the internal layer of the target tissue. The target tissue may be intact tissue, and the agent transfer surface may be configured to inhibit or prevent mechanical penetration of a surface of the target tissue when in contact with it during standard use of the device. “Standard use” of the device will be understood to comprise not applying the device with such force that the tissue in contact with the agent transfer surface or surrounding tissue is penetrated, pierced, or bruised. The target tissue may be skin. The target tissue may be mucosal tissue and the agent transfer surface may be configured to inhibit or prevent mechanical penetration of an intact epithelial layer of the mucosal tissue during standard use of the device.

In another embodiment, the present invention provides a method for inducing mucosal immunity in a subject, the method comprising:

contacting a target mucosal tissue of the subject with the agent transfer surface of a device of the present invention, and

applying an electrical signal to the electrode of the device to propagate an acoustic wave on and/or in the piezoelectric substrate of the device, and thereby deliver the agent through the agent transfer surface into the target mucosal tissue,

wherein delivery of the agent into the target mucosal tissue induces the mucosal immunity. The target mucosal tissue may be intact and the agent transfer surface may not penetrate an intact epithelial layer of the target mucosal tissue during standard use of the device. “Standard use” of the device will be understood to comprise not applying the device with such force that the tissue in contact with the agent transfer surface or surrounding tissue is penetrated, pierced, or bruised. It will be understood that different tissue types and locations thereof each have corresponding maximum standard use forces before penetration, piercing or bruising occurs.

In another as embodiment, the present invention provides an agent for use in a method of preventing or treating a disease in a subject, wherein the agent is present in a device comprising:

a piezoelectric substrate;

an agent carrier comprising an agent transfer surface for delivery of an agent into a tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode,

wherein the method comprises using the controller to apply the electrical signal to the electrode of the device to propagate an acoustic wave on and/or in the piezoelectric substrate, and thereby deliver the agent through the agent transfer surface into a target tissue to thereby prevent or treat the disease. The target tissue may be intact tissue and the agent transfer surface may be configured to inhibit or prevent mechanical penetration of a surface of the target tissue when in contact with it during standard use of the device. “Standard use” of the device will be understood to comprise not applying the device with such force that the tissue in contact with the agent transfer surface or surrounding tissue is penetrated, pierced, or bruised. The target tissue may be skin. The target tissue may be mucosal tissue and the agent transfer surface may be configured to inhibit or prevent mechanical penetration of an intact epithelial layer of the mucosal tissue during standard use of the device.

The device may be a device according to any embodiment of the present invention.

The subject referred to in the above embodiments may be suffering from the disease, may be exhibiting one or more symptoms of the disease, or may be capable of contracting the disease.

The methods referred to in the above embodiments may comprise delivering the agent into or through any one or more of: epithelium, sub-epithelium, mucosa, sub-mucosa, mucous membrane vasculature, nasal septum, cornea, corneal epithelium, Bowman's membrane, corneal stroma, corneal endothelium, conjunctiva, Tenon's fascia, episclera, sclera, choroid, choriocapillaris, Bruch's membrane, retinal pigment epithelium, neural retina, retinal blood vessels, internal limiting membrane, vitreous humour, skin epidermis, skin dermis, teeth and nails, a component of the gastro-intestinal system, a component of the genito-urinary, a component of the reproductive system (e.g. vagina, uterus), a component of the respiratory system, a component of the ocular system, a component of the auditory system, an eye, an ear, and a lip.

The methods referred to in the above embodiments may comprise using an acoustic frequency generator of the device to generate an acoustic frequency signal capable of modulating the primary acoustic excitation. The acoustic frequency signal may, for example, be in a range of 1 Hz to 100 kHz. For example, the primary acoustic excitation frequency may be more than 10⁶ Hz, more than 10⁷ Hz, more than 10⁸ Hz, more than 10⁹ Hz, more than 10¹⁰ Hz, or more than 10¹¹ Hz. The primary acoustic excitation frequency may be, for example, between 10⁶ Hz and 10⁷ Hz, between 10⁶ Hz and 10⁸ Hz, between 10⁶ Hz and 10⁹ Hz, between 10⁶ Hz and 10¹⁰ Hz, between 10⁷ Hz and 10⁸ Hz, between 10⁷ Hz and 10⁹ Hz, between 10⁷ Hz and 10¹⁰ Hz, between 10⁸ Hz and 10⁹ Hz, between 10⁸ Hz and 10¹⁰ Hz, or between 10⁹ Hz and 10¹⁰ Hz.

The methods referred to in the above embodiments may comprise delivering the agent into a target tissue that is one of: mammalian target tissue, human target tissue.

In another embodiment, the present invention provides use of an agent in the manufacture of a medicament for preventing or treating a disease in a subject, wherein the medicament is loaded in a device comprising:

a piezoelectric substrate;

an agent carrier comprising an agent transfer surface for delivery of an agent into a target tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the device into the tissue to thereby prevent or treat the disease.

In another embodiment, the present invention provides use of an agent in the manufacture of a medicament for preventing or treating a disease in a subject, wherein the medicament is prepared for use in a device comprising:

a piezoelectric substrate;

an agent carrier comprising an agent transfer surface for delivery of an agent into a target tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the device into the tissue to thereby prevent or treat the disease.

The device may be a device according to any embodiment of the present invention.

The medicament may be capable of inducing a mucosal immune response and/or a systemic immune response when administered through the device.

The agent of any of the above embodiments may comprise a solid or a combination of a solid and a liquid. The agent may comprise a solid comprising powder, granules, or a combination thereof. The agent may be lyophilised. The agent may comprise a therapeutic agent, a prophylactic agent, a diagnostic agent, a cosmetic agent, or any combination thereof. The agent may be selected from the group consisting of: a protein, a peptide, a polypeptide, an immunogenic agent, a vaccine, a biomimetic, a biosimilar, a biomaterial, a macromolecule, a small molecule, a sugar, a nucleic acid, an antibody, a drug, a nanoparticle, and any combination thereof.

The agent of any of the above embodiments may be delivered to a target depth within the tissue and/or at a specific rate of delivery using the controller of the device to regulate the duration, frequency and/or amplitude of the acoustic waves propagated on and/or in the piezoelectric substrate of the device. The acoustic wave amplitude may be in a range of 0.001 to 100 nm. The target depth may be in a range of 10 μm to 5 mm.

The electrical signal of any of the above embodiments may generate a primary acoustic excitation frequency on the piezoelectric substrate of the device in a range of 1 MHz to 100 GHz. For example, the primary acoustic excitation frequency may be more than 10⁶ Hz, more than 10⁷ Hz, more than 10⁸ Hz, more than 10⁹ Hz, more than 10¹⁰ Hz, or more than 10¹¹ Hz. The primary acoustic excitation frequency may be, for example, between 10⁶ Hz and 10⁷ Hz, between 10⁶ Hz and 10⁸ Hz, between 10⁶ Hz and 10⁹ Hz, between 10⁶ Hz and 10¹⁰ Hz, between 10⁷ Hz and 10⁸ Hz, between 10⁷ Hz and 10⁹ Hz, between 10⁷ Hz and 10¹⁰ Hz, between 10⁸ Hz and 10⁹ Hz, between 10⁸ Hz and 10¹⁰ Hz, or between 10⁹ Hz and 10¹⁰ Hz. The primary acoustic excitation frequency may correspond to the resonant frequency of the piezoelectric substrate.

Delivery of the agent into the target tissue according to any of the above embodiments may induce a mucosal immune response and/or a systemic immune response.

According to another embodiment of the present invention, there is provided a device, comprising:

a piezoelectric substrate;

a source of an agent on or acoustically couplable to the piezoelectric substrate; and

an electrode electrically couplable to the piezoelectric substrate;
a controller electrically couplable to the electrode and configured to apply an electrical signal to the electrode to propagate an acoustic wave on and/or in the piezoelectric substrate that is sufficient to deliver the agent from the source to under a surface of an area of tissue.

The piezoelectric substrate may comprise a single crystal piezoelectric material, a thin-film piezoelectric material, or any combination thereof.

The source may comprise the piezoelectric substrate, an absorbent, an adsorbent, a channel, a reservoir, a body, or any combination thereof.

The agent may comprise a liquid, a solid, a powder, or a combination thereof.

The agent may comprise a therapeutic agent, a prophylactic agent, a diagnostic agent, a cosmetic agent, or any combination thereof.

The electrode may comprise an interdigital transducer, a plate electrode, an electrode layer, or any combination thereof.

The acoustic wave may comprise a surface acoustic wave, a lamb wave, or a combination thereof.

The controller may be further configured to generate an ultrasonic wave to frequency modulate the surface acoustic wave, the lamb wave, or any combination thereof.

The area of tissue may comprise epithelial tissue, sub-epithelial tissue, or any combination thereof.

Another embodiment of the present invention provides a method, comprising non-invasively delivering an agent to a controllable depth under a surface of an area of tissue using the device described above.

The method may comprise a prophylactic method, a therapeutic method, a diagnostic method, a cosmetic method, or any combination thereof.

A further embodiment of the present invention provides a method, comprising conferring one or both of mucosal or systemic immunity by delivering an agent to epithelial or sub-epithelial tissue using the device described above.

Without limitation, it will be recognised that the present invention relates at least in part to the following listed exemplary embodiments:

### Embodiment 1

A device, comprising:

an agent carrier comprising an agent transfer surface for delivery of an agent into a tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the device into the tissue.

### Embodiment 2

The device of embodiment 1, wherein the piezoelectric substrate comprises a single crystal piezoelectric material, a thin-film piezoelectric material, or any combination thereof.

### Embodiment 3

The device of embodiment 1 or embodiment 2, wherein the piezoelectric substrate comprises any one or more of lithium niobate, tourmaline, single-crystal quartz, and/or lead zirconate titanate.

### Embodiment 4

The device of any one of embodiments 1 to 3, wherein the electrical signal generates a primary acoustic excitation frequency on and/or in the piezoelectric substrate in a range of 1 MHz to 10 GHz, more than 10⁶ Hz, more than 10⁷ Hz, more than 10⁸ Hz, more than 10⁹ Hz, more than 10¹⁰ Hz, or more than 10¹¹ Hz. The primary acoustic excitation frequency may be, for example, between 10⁶ Hz and 10⁷ Hz, between 10⁶ Hz and 10⁸ Hz, between 10⁶ Hz and 10⁹ Hz, between 10⁶ Hz and 10¹⁰ Hz, between 10⁷ Hz and 10⁸ Hz, between 10⁷ Hz and 10⁹ Hz, between 10⁷ Hz and 10¹⁰ Hz, between 10⁸ Hz and 10⁹ Hz, between 10⁸ Hz and 10¹⁰ Hz, or between 10⁹ Hz and 10¹⁰ Hz.

### Embodiment 5

The device of embodiment 4, wherein the primary acoustic excitation frequency corresponds to the resonant frequency of the piezoelectric substrate.

### Embodiment 6

The device of embodiment 4 or embodiment 5, further comprising an acoustic generator capable of generating a secondary acoustic excitation frequency capable of modulating the primary acoustic excitation.

### Embodiment 7

The device of embodiment 6, wherein the secondary acoustic excitation frequency is in a range of 1 Hz to 100 kHz.

### Embodiment 8

The device of any one of embodiments 5 to 7, wherein the controller is further configured to generate a secondary acoustic excitation to frequency modulate the primary acoustic excitation on and/or in the piezoelectric substrate.

### Embodiment 9

The device of any one of embodiments 1 to 8, wherein the acoustic wave is not a bulk acoustic wave or a Lamb wave.

### Embodiment 10

The device of any one of embodiments 1 to 9, wherein the acoustic wave is a surface acoustic wave.

### Embodiment 11

The device of any one of embodiments 1 to 10, wherein the acoustic wave is a Rayleigh surface acoustic wave.

### Embodiment 12

The device of any one of embodiments 1 to 11, wherein the device further comprises the agent.

### Embodiment 13

The device of any one of embodiments 1 to 12, wherein:

the agent carrier comprises the piezoelectric substrate,

the piezoelectric substrate comprises the agent transfer surface, and

the agent is present on the agent transfer surface.

### Embodiment 14

The device of embodiment 13, wherein:

the agent transfer surface is functionalised, and/or

the agent is lyophilised on the agent transfer surface,

to thereby retain the agent on the agent transfer surface.

### Embodiment 15

The device of any one of embodiments 1 to 14, wherein the agent carrier comprises any one or more of: an absorbent material, an adsorbent material, a micro channel, a reservoir, or any combination thereof.

### Embodiment 16

The device of any one of embodiments 1 to 15, wherein the agent carrier comprises a multiplicity of micro channels extending at least partially or wholly through the agent carrier to the agent transfer surface enabling retention of the agent and/or transportation of the agent to the tissue.

### Embodiment 17

The device of embodiment 16, wherein the agent carrier comprises a stack of layers, and the stack of layers comprises:

a first layer comprising the agent transfer surface; and

at least one other layer,

wherein holes formed in one layer of the plurality of layers are aligned with holes in an adjacent layer and in an arrangement facilitating a plurality of holes in a plurality of layers to cooperate to form the micro channels.

### Embodiment 18

The device according to embodiment 16 or embodiment 17, wherein the micro channels extend from the interior of the agent carrier body and terminate as pores at the agent transfer surface.

### Embodiment 19

The device according to any one of embodiments 16 to 18, wherein the agent transfer surface comprises a plurality of hollow micro protrusions in fluid communication with the micro channels.

### Embodiment 20

The device according to embodiment 19, wherein:

(i) the micro protrusions are not microneedles and do not function as microneedles; and

(ii) the tissue is intact tissue and the micro protrusions are shaped, arranged, and/or of a length that inhibits or prevents mechanical penetration of the surface of the tissue when the agent transfer surface is in contact with the tissue during standard use of the device.

### Embodiment 21

The device of embodiment 20, wherein the tissue is skin.

### Embodiment 22

The device of embodiment 21, wherein the tissue is mucosal tissue and the micro protrusions are shaped, arranged, and/or of a length that inhibits or prevents mechanical penetration of an intact epithelial layer of the mucosal tissue during standard use of the device.

### Embodiment 23

The device according to any one of embodiments 1 to 22, comprising or in fluid communication with one or more reservoirs of the agent.

### Embodiment 24

The device of embodiment 23, wherein the agent reservoirs comprise a void formed within the agent carrier body.

### Embodiment 25

The device of embodiment 23 or embodiment 24, wherein the agent transfer surface comprises a plurality of protrusions in fluid communication with the agent reservoirs.

### Embodiment 26

The device of embodiment 25, wherein the plurality of protrusions extend outward from an inside of one or more of the voids and terminate at the agent transfer surface.

### Embodiment 27

The device of embodiment 26, wherein one or more of the voids is formed by a peripheral structure, and:

(i) the peripheral structure terminates in a common plane with the plurality of protrusions; or

(ii) the plurality of protrusions extend outward from the void beyond the peripheral structure; or

(iii) the plurality of protrusions terminate in a plane and the peripheral structure terminates short of the plane such that the plurality of protrusions extend beyond the peripheral structure.

### Embodiment 28

The device of any one of embodiments 23 to 27, wherein:

(i) the plurality of protrusions are not microneedles and do not function as microneedles; and

(ii) the tissue is intact tissue and the plurality of protrusions are shaped, arranged, and/or of a length that inhibits or prevents mechanical penetration of the surface of the tissue when the agent transfer surface is in contact with the tissue during standard use of the device.

### Embodiment 29

The device of embodiment 28, wherein the tissue is skin.

### Embodiment 30

The device of embodiment 28, wherein the tissue is mucosal tissue and the plurality of protrusions are shaped, arranged, and/or of a length that inhibits or prevents mechanical penetration of an intact epithelial layer of the mucosal tissue during standard use of the device

### Embodiment 31

The device of any one of embodiments 1 to 30, wherein the agent carrier is formed from any one or more of a polymer, a metal, silicon, a ceramic, and/or plastic.

### Embodiment 32

The device of any one of embodiments 1 to 31, wherein the agent comprises a solid or a combination of a solid and a liquid.

### Embodiment 33

The device of any one of embodiments 1 to 32, wherein the agent comprises a solid comprising powder, granules, or any combination thereof.

### Embodiment 34

The device of any one of embodiments 1 to 33, wherein the agent is lyophilised.

### Embodiment 35

The device of any one of embodiments 1 to 34, wherein the agent comprises a therapeutic agent, a prophylactic agent, a diagnostic agent, a cosmetic agent, or any combination thereof.

### Embodiment 36

The device of any one of embodiments 1 to 35, wherein the agent is selected from the group consisting of: a protein, a peptide, a polypeptide, an immunogenic agent, a vaccine, a biomimetic, a biosimilar, a biomaterial, a macromolecule, a small molecule, a sugar, a nucleic acid, an antibody, a drug, a nanoparticle, and any combination thereof.

### Embodiment 37

A method for delivering an agent to an internal layer within a target tissue, the method comprising:

contacting the target tissue with the agent transfer surface of the device of any one of embodiments 1 to 36, and

applying an electrical signal to the electrode of the device to propagate an acoustic wave on and/or in the piezoelectric substrate of the device, and thereby deliver the agent through the agent transfer surface to the internal layer of the target tissue.

### Embodiment 38

The method of embodiment 37, wherein:

the target tissue is intact tissue, and

the agent transfer surface is configured to inhibit or prevent mechanical penetration of a surface of the target tissue when in contact with it during standard use of the device.

### Embodiment 39

The method of embodiment 38, wherein the target tissue is skin.

### Embodiment 40

The method of embodiment 38, wherein the target tissue is mucosal tissue and the agent transfer surface is configured to inhibit or prevent mechanical penetration of an intact epithelial layer of the mucosal tissue during standard use of the device.

### Embodiment 41

A method for inducing mucosal immunity in a subject, the method comprising:

contacting a target mucosal tissue of the subject with the agent transfer surface of the device of any one of embodiments 1 to 36, and

applying an electrical signal to the electrode of the device to propagate an acoustic wave on and/or in the piezoelectric substrate of the device, and thereby deliver the agent through the agent transfer surface into the target mucosal tissue.

wherein delivery of the agent into the target mucosal tissue induces the mucosal immunity.

### Embodiment 42

The method of embodiment 41, wherein:

the target mucosal tissue is intact, and

the agent transfer surface does not penetrate an intact epithelial layer of the target mucosal tissue during standard use of the device.

### Embodiment 43

An agent for use in a method of preventing or treating a disease in a subject, wherein the agent is present in a device comprising:

a piezoelectric substrate;

an agent carrier comprising an agent transfer surface for delivery of an agent into a tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode,

wherein the method comprises using the controller to apply the electrical signal to the electrode of the device to propagate an acoustic wave on and/or in the piezoelectric substrate, and thereby deliver the agent through the agent transfer surface into a target tissue to thereby prevent or treat the disease.

### Embodiment 44

The agent of embodiment 43, wherein:

the target tissue is intact tissue, and

the agent transfer surface is configured to inhibit or prevent mechanical penetration of a surface of the target tissue when in contact with it during standard use of the device.

### Embodiment 45

The agent of embodiment 44, wherein the target tissue is skin.

### Embodiment 46

The agent of embodiment 44, wherein the target tissue is mucosal tissue and the agent transfer surface is configured to inhibit or prevent mechanical penetration of an intact epithelial layer of the mucosal tissue during standard use of the device.

### Embodiment 47

The agent of any one of embodiments 43 to 46, wherein the device is a device according to any one of embodiments 1 to 36.

### Embodiment 48

The method of any one of embodiments 37 to 42, or the agent of any one embodiments 43 to 47, wherein the subject is suffering from the disease, is exhibiting one or more symptoms of the disease, or is capable of contracting the disease.

### Embodiment 49

The method of any one of embodiments 37 to 42, or 48, or the agent of any one of embodiments 43 to 48, wherein delivery of the agent into the target tissue induces a mucosal immune response and/or a systemic immune response.

### Embodiment 50

The method of any one of embodiments 37 to 42, 48 or 49, or the agent of any one of embodiments s 43 to 49, wherein the method comprises delivering the agent into or through any one or more of: epithelium, sub-epithelium, mucosa, sub-mucosa, mucous membrane vasculature, nasal septum, cornea, corneal epithelium, Bowman's membrane, corneal stroma, corneal endothelium, conjunctiva, Tenon's fascia, episclera, sclera, choroid, choriocapillaris, Bruch's membrane, retinal pigment epithelium, neural retina, retinal blood vessels, internal limiting membrane, vitreous humour, skin epidermis, skin dermis, teeth and nails, a component of the gastro-intestinal system, a component of the genito-urinary, a component of the reproductive system (e.g. vagina, uterus), a component of the respiratory system, a component of the ocular system, a component of the auditory system, an eye, an ear, and a lip.

### Embodiment 51

The method of any one of embodiments 37 to 42, or 48 to 50, or the agent of any one of embodiments 43 to 50, wherein the agent is delivered to a target depth within the tissue and/or at a specific rate of delivery using the controller of the device to regulate the duration, frequency and/or amplitude of the acoustic waves propagated on and/or in the piezoelectric substrate of the device.

### Embodiment 52

The method or agent of embodiment 51, wherein the acoustic wave amplitude is in a range of 0.001 to 100 nm.

### Embodiment 53

The method or agent of embodiment 51 or embodiment 52, wherein the target depth is in a range of 10 μm to 5 mm.

### Embodiment 54

The method of any one of embodiments 37 to 42, or 48 to 53, or the agent of any one of embodiments 43 to 53, wherein the electrical signal generates a primary acoustic excitation frequency on the piezoelectric substrate of the device in a range of 1 MHz to 10 GHz more than 10⁶ Hz, more than 10⁷ Hz, more than 10⁸ Hz, more than 10⁹ Hz, more than 10¹⁰ Hz, more than 10¹¹ Hz, between 10⁶ Hz and 10⁷ Hz, between 10⁶ Hz and 10⁸ Hz, between 10⁶ Hz and 10⁹ Hz, between 10⁶ Hz and 10¹⁰ Hz, or between 10⁶ Hz and 10¹¹ Hz.

### Embodiment 55

The method or agent of embodiment 54, wherein the primary acoustic excitation frequency corresponds to the resonant frequency of the piezoelectric substrate.

### Embodiment 56

The method or agent of embodiment 54 or embodiment 55, comprising using an acoustic frequency generator of the device to generate an acoustic frequency signal capable of modulating the acoustic excitation.

### Embodiment 57

The method or agent of embodiment 56, wherein the acoustic frequency signal is in a range of 1 Hz to 100 kHz.

### Embodiment 58

The method of any one of embodiments 37 to 42, or 48 to 57, or the agent of any one of embodiments 43 to 57, wherein the agent is selected from the group consisting of: a protein, a peptide, a polypeptide, an immunogenic agent, a vaccine, a biomimetic, a biosimilar, a biomaterial, a macromolecule, a small molecule, a sugar, a nucleic acid, an antibody, drugs, a nanoparticle, and any combination thereof.

### Embodiment 59

The method of any one of embodiments 37 to 42, or 48 to 58, or the agent of any one of embodiments 43 to 58, wherein the target tissue is one of: mammalian target tissue, human target tissue.

### Embodiment 60

Use of an agent in the manufacture of a medicament for preventing or treating a disease in a subject, wherein the medicament is loaded in a device comprising:

a piezoelectric substrate;

an agent carrier comprising an agent transfer surface for delivery of an agent into a target tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the device into the tissue to thereby prevent or treat the disease.

### Embodiment 61

Use of an agent in the manufacture of a medicament for preventing or treating a disease in a subject, wherein the medicament is prepared for use in a device comprising:

a piezoelectric substrate;

an agent carrier comprising an agent transfer surface for delivery of an agent into a target tissue, wherein the agent carrier comprises or is electrically couplable to a piezoelectric substrate;

an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the device into the tissue to thereby prevent or treat the disease.

### Embodiment 62

The use of embodiment 60 or embodiment 61, wherein the device is a device according to any one of embodiments 1 to 29.

### Embodiment 63

The use of any one of embodiments 60 to 62, wherein the agent is selected from the group consisting of: a protein, a peptide, a polypeptide, an immunogenic agent, a vaccine, a biomimetic, a biosimilar, a biomaterial, a macromolecule, a small molecule, a sugar, a nucleic acid, an antibody, drugs, a nanoparticle, and any combination thereof.

### Embodiment 64

The use of any one of embodiments 60 to 63, wherein the medicament is capable of inducing a mucosal immune response and/or a systemic immune response when administered through the device.

### Embodiment 65

A method for delivering a target amount of agent to an internal layer within a target tissue, the method comprising:

contacting the target tissue surface with the agent transfer surface of the device of any one of embodiments 1 to 36, and

applying an electrical signal to the electrode of the device to propagate an acoustic wave on and/or in the piezoelectric substrate of the device, to:
transport the agent through the agent carrier to the agent transfer surface
enhance the permeability of the target tissue surface.

### Embodiment 66

The method of embodiment 65, wherein the method comprises delivering the agent into or through any one or more of: epithelium, sub-epithelium, mucosa, sub-mucosa, mucous membrane vasculature, nasal septum, cornea, corneal epithelium, Bowman's membrane, corneal stroma, corneal endothelium, conjunctiva, Tenon's fascia, episclera, sclera, choroid, choriocapillaris, Bruch's membrane, retinal pigment epithelium, neural retina, retinal blood vessels, internal limiting membrane, vitreous humour, teeth, a component of the gastro-intestinal system, a component of the genito-urinary, a component of the reproductive system (e.g. vagina, uterus), a component of the respiratory system, a component of the ocular system, a component of the auditory system, an eye, an ear, and a lip.

### Embodiment 67

The method of embodiment 65 or embodiment 66, wherein:

the target tissue is intact tissue, and

the agent transfer surface is configured to inhibit or prevent mechanical penetration of a surface of the target tissue when in contact with it during standard use of the device.

### Embodiment 68

The method of any one of embodiments 65 to 67, wherein the target tissue is mucosal tissue, or the eye.

### Embodiment 69

The method of embodiment 68, wherein the target mucosal tissue is intact, the agent transfer surface does not penetrate an intact epithelial layer of the target mucosal tissue during standard use of the device, and wherein delivery of a therapeutically effective amount of the agent into the target mucosal tissue induces mucosal immunity.

### Embodiment 70

The method of embodiment 68, wherein the target tissue is the eye, and the method comprises contacting the agent transfer surface with corneal epithelium, and/or corneal limbus and delivering a target amount of the agent into the cornea of the eye.

### Embodiment 71

The method of embodiment 70, wherein:

the agent is delivered for the treatment of myopia or keratoconus,

the target amount of the agent is a therapeutically effective amount of any one or more of riboflavin-5-phosphate-sodium, Glutaraldehyde, Grape seed extract, and/or Genipin, and

the method further comprises exposing the cornea to ultraviolet light following delivery of the therapeutic amount of the agent to the cornea for a time period sufficient to induce collagen crosslinking in the cornea.

### Embodiment 72

The method of embodiment 71, further comprising repeating the delivery of the therapeutically effective amount and the exposure to ultraviolet light within 1, 2, 3, 4, 5, 6, 7, 14, 21, 28, 42 or 60 days.

### Embodiment 73

The method of embodiment 70, wherein:

the target amount of the agent comprises a therapeutic amount of the agent for treating a condition or disease upon delivery to the posterior segment of the eye, and the therapeutically effective amount of the agent
is delivered through the corneal epithelium, Bowman's membrane, Corneal stroma and Corneal endothelium, into aqueous humor,
circulates within the aqueous humor through the pupil and around the lens into the posterior chamber,
contacts one or more of: vitreous humor, ciliary body blood vessels, uveal blood vessels in the pars plana, and
is distributed via the choroidal vasculature to the posterior segment of the eye.

### Embodiment 74

The method of embodiment 68, wherein:

the target amount of the agent comprises a therapeutic amount of the agent for treating a condition or disease upon delivery to the posterior segment of the eye,

and the therapeutically effective amount of the agent
is delivered through the conjunctiva overlying the sclera, and the sclera,
enters the uveal tract of the eye,
is distributed via the choroidal vasculature to the choroid and retina in the posterior segment of the eye.

Again without limitation, it will be recognised that the present invention relates at least in part to the following listed exemplary embodiments:

### Embodiment 1

A device, comprising:

an agent carrier comprising an agent transfer surface for delivery of an agent into a tissue, wherein the agent carrier comprises or is acoustically couplable to a piezoelectric substrate;

an electrode electrically couplable to the piezoelectric substrate; and

a controller electrically couplable to the electrode and configured to apply an electrical signal to the electrode to propagate an acoustic wave on and/or in the piezoelectric substrate which is capable of delivering the agent from the device into the tissue.

### Embodiment 2

The device of embodiment 1, wherein the controller is configured to apply the electrical signal at a level which generates a primary acoustic excitation frequency on and/or in the piezoelectric substrate of more than 10⁶ Hz, between 10⁶ Hz and 10⁷ Hz, between 10⁶ Hz and 10⁸ Hz, between 10⁶ Hz and 10⁹ Hz, or between 10⁶ Hz and 10¹⁰ Hz.

### Embodiment 3

The device of embodiment 1 or embodiment 2, further comprising an acoustic generator capable of generating a secondary acoustic excitation frequency capable of modulating a primary acoustic excitation frequency generated by the piezoelectric substrate, wherein the secondary acoustic excitation frequency is less than or equal to the primary acoustic excitation frequency.

### Embodiment 4

The device of any one of embodiments 1 to 3, wherein the acoustic wave is a surface acoustic wave (e.g. a Rayleigh surface acoustic wave).

### Embodiment 5

The device of any one of embodiments 1 to 4, wherein the device:

does not comprise an electrode for contacting the tissue surface, and/or

is not configured to utilise repulsive electromotive force to transport a charged agent (e.g. therapeutically effective amount of a charged agent) into and/or through the tissue in contact with the agent transfer surface, and/or

is incapable of generating or maintaining a difference in electric potential between the agent transfer surface of the device and the tissue surface in contact with it to consequently induce transport of the agent from the device into the tissue, and/or

is incapable of utilising repulsive electromotive force to transport a charged agent into and/or through the tissue in contact with the agent transfer surface, and/or

is incapable of permeating the tissue by any of iontophoresis (ionization), ionophoresis, electrophoresis, microelectrophoresis, electroosmosis, cataphoresis, electroendosmosis, and electrorepulsion.

### Embodiment 6

The device of any one of embodiments 1 to 5, wherein:

the agent carrier comprises the piezoelectric substrate,

the piezoelectric substrate comprises the agent transfer surface, and

the agent is present on the agent transfer surface, and is optionally functionalised and/or lyophilised on the agent transfer surface.

### Embodiment 7

The device of any one of embodiments 1 to 6, wherein:

(i) the agent carrier comprises a multiplicity of micro channels extending partially or wholly through the agent carrier to the agent transfer surface enabling retention of the agent and/or transportation of the agent to the tissue; and

(ii) the micro channels extend from the interior of the agent carrier body and terminate as pores at the agent transfer surface, and/or the agent transfer surface comprises a plurality of hollow micro protrusions in fluid communication with the micro channels; and

(iii) the micro protrusions are not microneedles and do not function as microneedles.

### Embodiment 8

The device according to any one of embodiments 1 to 6, comprising or in fluid communication with one or more reservoirs of the agent, wherein:

(i) the agent reservoirs comprise a void formed within the agent carrier body; and

(ii) the agent transfer surface comprises a plurality of protrusions in fluid communication with the agent reservoirs; and

(iii) optionally the plurality of protrusions extend outward from an inside of one or more of the voids and terminate at the agent transfer surface; and

(iv) the protrusions are not microneedles and do not function as microneedles.

### Embodiment 9

The device of embodiment 8, wherein one or more of the voids is formed by a peripheral structure, and:

(i) the peripheral structure terminates in a common plane with the plurality of protrusions; or

(ii) the plurality of protrusions extend outward from the void beyond the peripheral structure; or

(iii) the plurality of protrusions terminate in a plane and the peripheral structure terminates short of the plane such that the plurality of protrusions extend beyond the peripheral structure.

### Embodiment 10

A method for delivering an agent to an internal layer within a target tissue, the method comprising:

contacting the target tissue with the agent transfer surface of the device of any one of embodiments 1 to 9, and

applying an electrical signal to the electrode of the device to propagate an acoustic wave on and/or in the piezoelectric substrate of the device, and thereby deliver the agent through the agent transfer surface to the internal layer of the target tissue.

### Embodiment 11

The method of embodiment 10, wherein the method comprises delivering the agent into or through any one or more of: epithelium, sub-epithelium, mucosa, sub-mucosa, mucous membrane vasculature, nasal septum, cornea, corneal epithelium, Bowman's membrane, corneal stroma, corneal endothelium, conjunctiva, Tenon's fascia, episclera, sclera, choroid, choriocapillaris, Bruch's membrane, retinal pigment epithelium, neural retina, retinal blood vessels, internal limiting membrane, vitreous humour, teeth, a component of the gastro-intestinal system, a component of the genito-urinary, a component of the reproductive system (e.g. vagina, uterus), a component of the respiratory system, a component of the ocular system, a component of the auditory system, an eye, an ear, and a lip.

### Embodiment 12

The method of embodiment 10 or embodiment 11, wherein:

the target tissue is intact tissue, and

the agent transfer surface is configured to inhibit or prevent mechanical penetration of a surface of the target tissue when in contact with it during standard use of the device.

### Embodiment 13

The method of any one of embodiments 10 to 12, wherein the target tissue is mucosal tissue, or the eye.

### Embodiment 14

The method of embodiment 13, wherein the target mucosal tissue is intact, the agent transfer surface does not penetrate an intact epithelial layer of the target mucosal tissue during standard use of the device, and wherein delivery of a therapeutically effective amount of the agent into the target mucosal tissue induces mucosal immunity.

### Embodiment 15

The method of embodiment 13, wherein the target tissue is the eye, and the method comprises contacting the agent transfer surface with corneal epithelium, and delivering a target amount of the agent into the cornea of the eye.

### Embodiment 16

The method of embodiment 15, wherein:

the agent is delivered for the treatment of myopia or keratoconus,

the agent is a therapeutically effective amount of any one or more of Riboflavin-5-phosphate-sodium, Glutaraldehyde, Grape seed extract, and/or Genipin, and

the method further comprises exposing the cornea to ultraviolet light following delivery of the therapeutic amount of the agent to the cornea for a time period sufficient to induce collagen crosslinking in the cornea.

### Embodiment 17

The method of embodiment 16, further comprising repeating the delivery of the therapeutically effective amount and the exposure to ultraviolet light within 1, 2, 3, 4, 5, 6, 7, 14, 21, 28, 42 or 60 days.

### Embodiment 18

The method of embodiment 15, wherein:

the agent comprises a therapeutic amount of the agent for treating a condition or disease upon delivery to the posterior segment of the eye,

and the therapeutically effective amount of the agent
is delivered through the corneal epithelium, Bowman's membrane, Corneal stroma, Descemet's membrane and Corneal endothelium, into aqueous humor,
circulates within the aqueous humor through the pupil and around the lens into the posterior chamber,
contacts one or more of: vitreous humor, ciliary body blood vessels, uveal blood vessels in the pars plana, and
is distributed via the choroidal vasculature to the posterior segment of the eye.

### Embodiment 19

The method of embodiment 13, wherein:

the the agent comprises a therapeutic amount of the agent for treating a condition or disease upon delivery to the posterior segment of the eye,

and the therapeutically effective amount of the agent
is delivered through the conjunctiva overlying the sclera, and the sclera,
enters the uveal tract of the eye,
is distributed via the choroidal vasculature to the choroid and retina in the posterior segment of the eye.

### Embodiment 20

The method of embodiment 18 or embodiment 19, wherein the therapeutically effective amount of the agent comprises anti-Vascular Endothelial Growth Factor (anti-VEGF) agents, nucleic acids, and/or an anti-inflammatory drug, and is delivered for the treatment of Age Related Macular Degeneration, Diabetic Eye Disease, or Posterior Choroiditis.

### BRIEF DESCRIPTION OF DRAWINGS

Embodiments of the invention will now be described by way of example only with reference to the accompanying drawings, in which:

FIG. 1 is a schematic diagram of a first embodiment of a device where a surface acoustic wave (SAW) is applied to an absorbent containing the agent for drug delivery according to the present invention;

FIG. 2 is a schematic diagram of a second embodiment of the device where a bulk acoustic wave (BAW) is applied to an absorbent containing the agent for drug delivery according to the present invention;

FIG. 3 is a schematic diagram of a third embodiment of the device where a SAW is applied to an absorbent containing the agent that is in contact with a superstrate for drug delivery according to the present invention;

FIG. 4 is a schematic diagram of a fourth embodiment of the device where a SAW is applied to a solid having microfabricated features on it that contains the agent for drug delivery according to the present invention;

FIG. 5 is a schematic diagram of a fifth embodiment of the device where a SAW is applied to a fluid couplant in contact with a solid having microfabricated features on it that contains the agent for drug delivery according to the present invention;

FIG. 6 is a schematic diagram of a sixth embodiment of the device where a BAW is applied to a solid having microfabricated features on it that contains the agent for drug delivery according to the present invention; and

FIG. 7 is a graph of pixel intensity versus depth for FITC-albumin delivered by the SAW device of FIG. 1 and a control.

FIG. 8 (A-Q) is a series of diagrams depicting a device and components thereof according to embodiments of the present invention. A=top view of the housing of a device. B=exploded view of the device illustrating the ultrasonic housing, location of the piezo electric transducer and agent carrier. C=top view of an agent carrier and agent carrier body that has a 5 mm circumference agent transfer surface. D=cross section view of an agent carrier and agent carrier body that has a 5 mm circumference agent transfer surface. The agent carrier can be filled with agent via the male leur port. E=cross section view of an agent carrier and agent carrier body that has a 5 mm circumference agent transfer surface. The agent carrier can be filled with agent via the female syringe port. F=top view of an agent carrier and agent carrier body that has a 7 mm circumference agent transfer surface. G=cross section view of an agent carrier and agent carrier body that has a 7 mm circumference agent transfer surface. The agent carrier can be filled with agent via the male leur port. H=cross section view of an agent carrier and agent carrier body that has a 5 mm circumference agent transfer surface. The agent carrier can be filled with agent via the female syringe port. I=top view of 50 micron wide hexagonally shaped micro channels extending wholly through the agent carrier to the agent transfer surface. The micro channels are arranged in a pattern where they are 22 microns apart from each other. J=side view of an agent carrier. K=diagonal side view of an agent carrier. L=detailed volumetric cross section view of an agent carrier and agent carrier body that has a 7 mm circumference agent transfer surface. The agent carrier can be filled with agent via the male leur port. M=detailed top view of an agent carrier and agent carrier body that has a 7 mm circumference agent transfer surface. N=side view of an agent carrier and agent carrier body. The agent carrier can be filled with agent via the male leur port. O=Exploded diagonal side view of an agent carrier and piezo electric transducer. P=top view of 50 micron wide hexagonally shaped micro channels extending wholly through the agent carrier to the agent transfer surface. The micro channels are arranged in a pattern where they are 17 microns apart from each other.

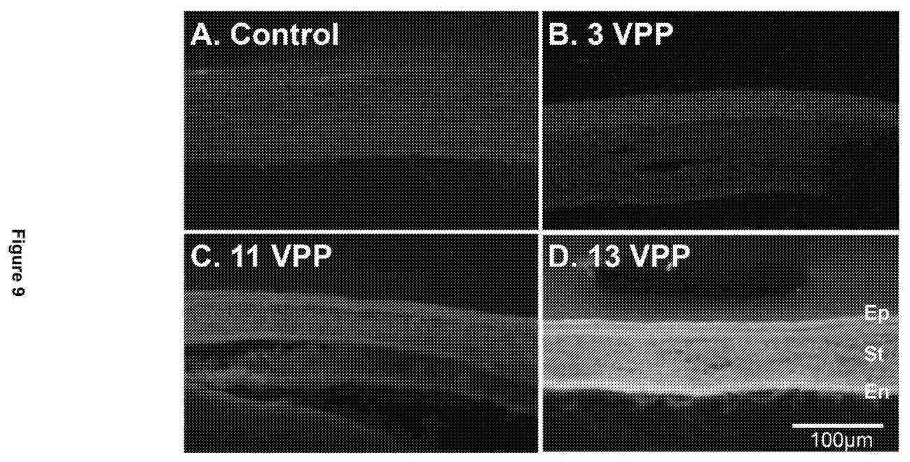

FIG. 9 shows fluorescence microscopy images generated from the testing of various voltages for delivery of Riboflavin-5-phosphate-sodium to the rat cornea using an exemplary device of the present invention described in Example 2 and illustrated in FIG. 8. In both the control (A) and 3 VPP (B) very little Riboflavin-5-phosphate-sodium was observable in the cornea. However, at 11 VPP (C) and 13 VPP (D) Riboflavin-5-phosphate-sodium was detectable. EP=epithelium, St=stroma and En=endothelium. Scale shown is 100 μm.

FIG. 10 (A-C) shows fluorescence microscopy images generated upon attempted delivery of 0.2% delivery of Riboflavin-5-phosphate-sodium to the cornea using an exemplary device of the present invention described in Example 2 and illustrated in FIG. 8. No detection of fluorescence was evident above background levels (A) in the control cornea (B). Animals exposed to 13 VPP and 0.2% Riboflavin-5-phosphate-sodium showed increased but variable fluorescence (C, D). Quantification of the Riboflavin-5-phosphate-sodium (E, F) showed an increase in the treated group (G) with strong statistical significance (P<0.0001, n=3) via unpaired student's t-test. Scale shown is 100 μm.

FIG. 11 shows fluorescence microscopy images of whole eye visualisation of 0.2% Riboflavin-5-phosphate-sodium delivered to the cornea using an exemplary device of the present invention described in Example 2 and illustrated in FIG. 8. Fluorescence was only detected at background levels in the control eyes (A). Using the device at 13 VPP, increased Riboflavin-5-phosphate-sodium fluorescence was detected in the cornea, retina, limbus, sclera, choroid and lens at variable levels (B-D). A significant amount of riboflavin-5-phosphate-sodium was delivered to the Choroid. Scale shown is 1 mm.

FIG. 12 is a schematic diagram depicting a device and components thereof according to embodiments of the present invention in contact with a tissue surface. A=pig lip tissue, B=FITC-albumin/Fluorescein in tris-HCL buffer, C=lithium niobate (piezoelectric substrate).

FIG. 13 provides graphs showing (A) the effect of time on FITC-Albumin perfusion, (B) the effect of SAW power on FITC-Albumin perfusion, (C) the effect of time on fluorescein perfusion, and (D) the effect of SAW power on fluorescein perfusion in pig lip tissue using a device according to embodiments of the present invention.

FIG. 14 shows Hematoxylin and eosin staining of pig lip sections treated with a device according to embodiments of the present invention. A=control (no SAW exposure), B=SAW exposed tissue for 5 seconds and 50 mV.

### DESCRIPTION OF EMBODIMENTS

The present invention provides a device for the non-invasive delivery of an agent into tissue. The devices propagate an acoustic wave on and/or in a piezoelectric substrate which is used as a transportation stimulus to move the agent through the device and deliver it into the tissue. By way of non-limiting example, the acoustic wave may be a surface acoustic wave (SAW). The agent transfer surface of the device does not mechanically penetrate or destroy any layer of tissue to which it is applied, and can thus deliver an agent into tissue in a non-invasive manner. In this specification, the term “non-invasive” will be understood to mean a method of delivering an agent to tissue that does not mechanically penetrate, pierce or destroy any layer of tissue.

### Devices

Devices according to the present invention generally comprise an agent carrier which may comprise or be acoustically couplable to a piezoelectric substrate. The agent carrier also comprises an agent transfer surface for delivery of an agent into a tissue. The devices may also comprise an electrode electrically couplable to the piezoelectric substrate and a controller electrically couplable to the electrode. The controller may be configured to apply an electrical signal to the electrode to propagate an acoustic wave on and/or in the piezoelectric substrate which is capable of delivering the agent from the agent transfer surface into the tissue.

The acoustic wave may temporarily increase the permeability of a tissue in contact with the agent transfer surface of the device to thereby facilitate the entry of agent into the tissue. Without being limited by theory, the mechanisms for the entry of the agent into the tissue may include cavitation, fluidic jetting, physical vibration of cells making their surface membranes more permeable, and stretching the inter-cellular spaces and cell to cell complexes whose adhesions hold adjacent cell walls together. The agent loaded in the device may be transported by, released by, and actively delivered into and/or through the tissue solely by virtue of the acoustic wave generated during operation of the device.

Referring to the drawings a device 10 for drug delivery according to embodiments of the present invention may generally comprise an electroacoustic transducer 12 on a piezoelectric substrate 14. The electroacoustic transducer 12 may be controlled by a controller (not shown). A source 16 of an agent may be fluidly couplable to, or physically contactable with, tissue 18, and acoustically couplable to the piezoelectric substrate 14. Further or alternatively, the source 16 of the agent may comprise the piezoelectric substrate 14 itself (i.e., the agent is disposed directly on the piezoelectric substrate 14). The source 16 may comprise a fluid comprising the agent, for example, a liquid containing the therapeutic agent.

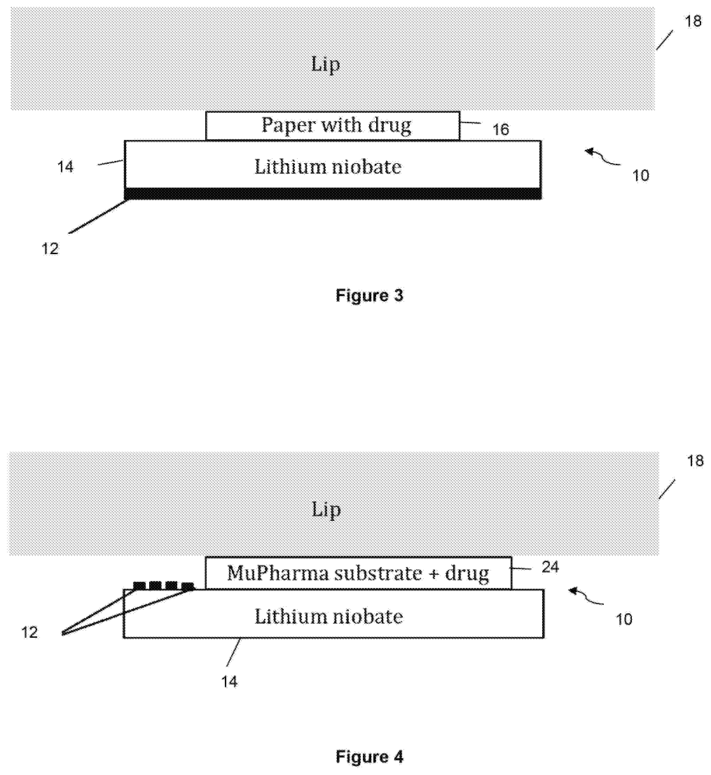

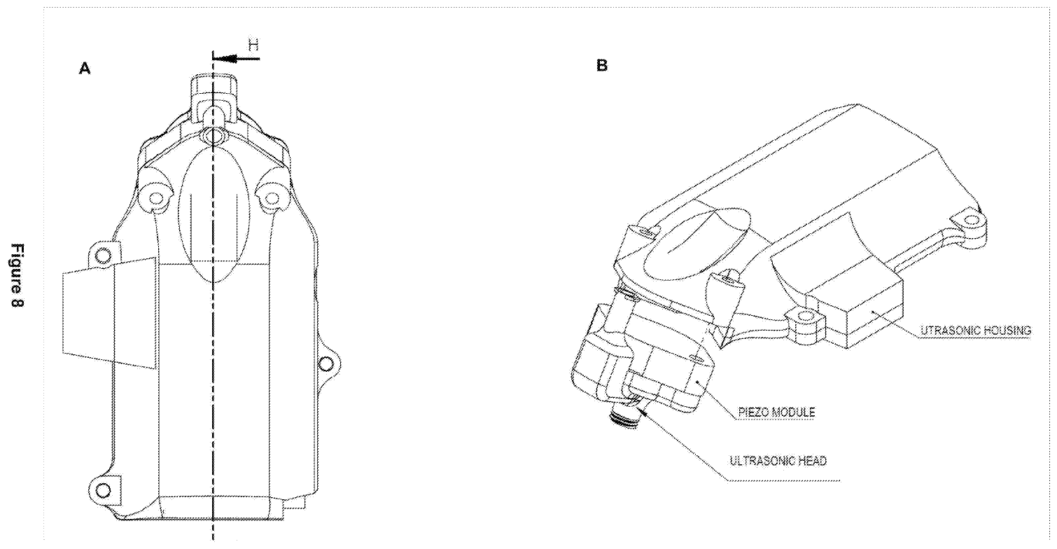

Referring to FIG. 5, one embodiment of the device 10 may further comprise a fluid couplant 22 interposed between the fluid source 16 and the piezoelectric substrate 14. Alternatively, as illustrated in FIG. 2, another embodiment of the agent apparatus 10 may further comprise a superstrate 20 interposed between the fluid couplant 22 and the fluid source 16.

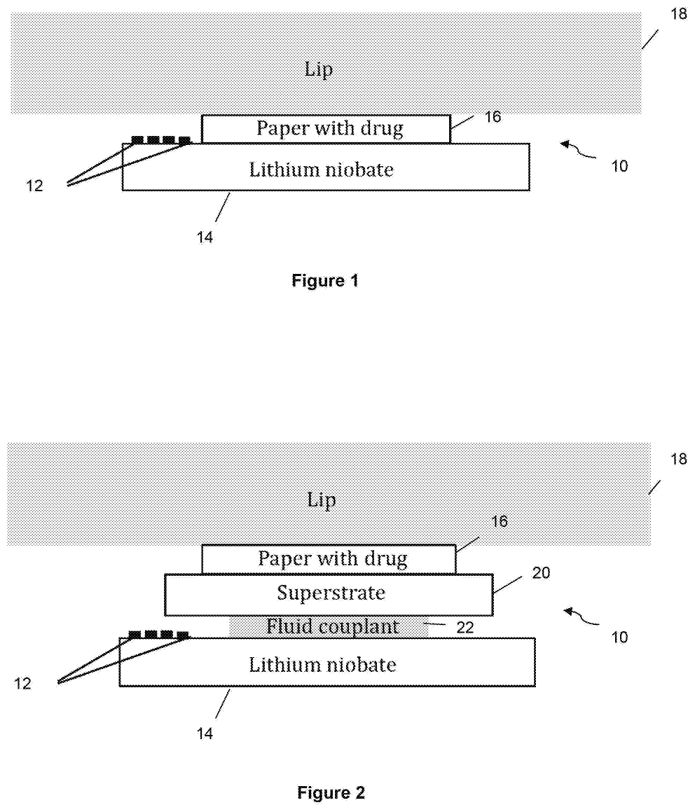

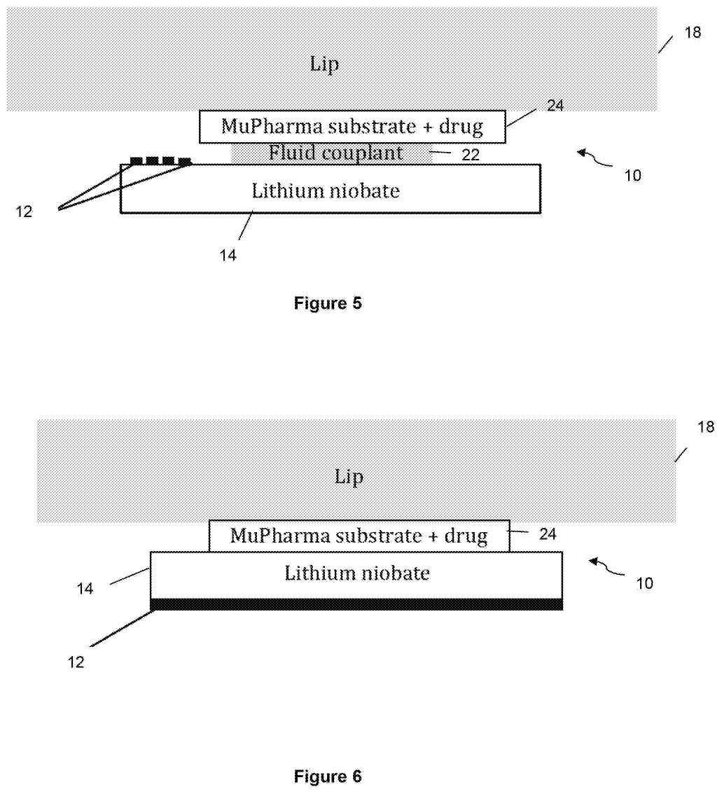

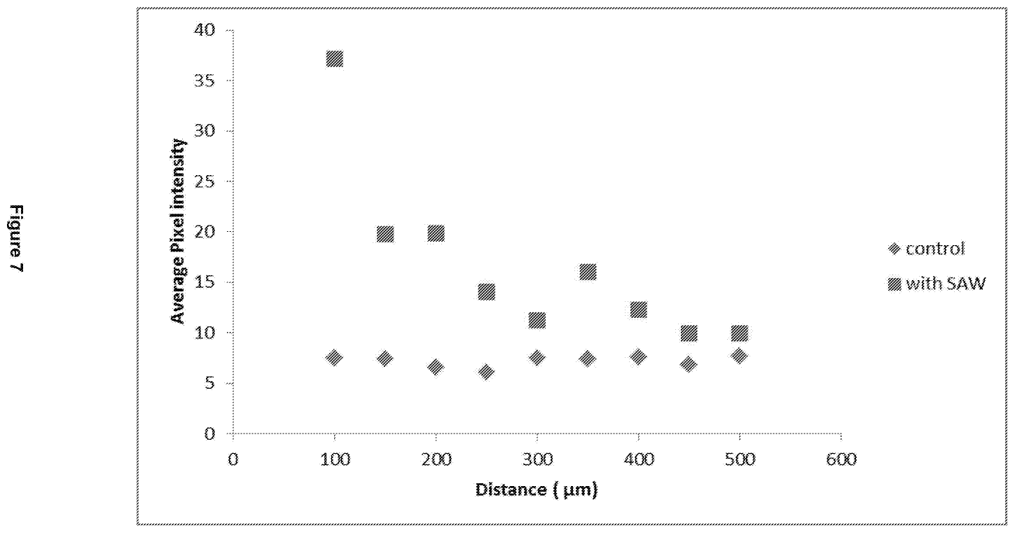

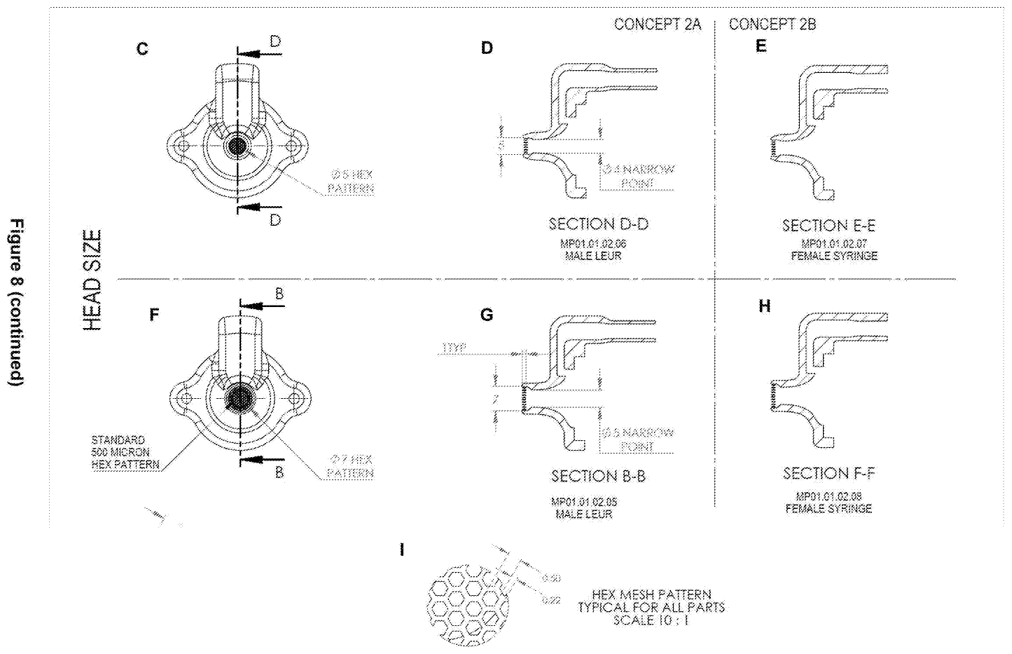

The electroacoustic transducer 12 may comprise interdigital transducers (IDTs), plate electrode, or an electrode layer. The piezoelectric substrate 14 may, for example, comprise a lithium niobate (LiNbO₃) substrate. The controller may, for example, be a programmable microcontroller. As illustrated in FIGS. 1 to 3, agent 16 may be contained in an absorbent, such as paper. Alternatively, the agent 16 may not be contained in an absorbent. In some embodiments the agent 16 may not be contained in paper. As illustrated in FIGS. 4 to 6, the agent 16 may be contained in a reservoir, such as a microfluidic reservoir or fluid micro channels formed on a silicon substrate. The micro channels may extend from a reservoir of the agent partially or wholly through the device. Other alternative or equivalent materials, components and arrangements may also be used for the electroacoustic transducer 12, the piezoelectric substrate 14, the fluid source 16, and the controller.

The controller of the device 10 may be configured to apply signals (which may include RF signals) to the electroacoustic transducer 12 to controllably generate acoustic waves that fluidly couple with and drivingly transport, the agent to controllably deliver the agent to and into tissue 18. The controllable delivery of the agent across an epithelial membrane 18 may elicit a systemic immune response or a mucosal immune response (or both) in a subject. Preferably, at least a mucosal immune response is induced, and optionally a systemic immune response is also induced.

The acoustic waves generated by the device 10 may have a frequency corresponding to the resonant frequency of the piezoelectric substrate 14. The acoustic waves may comprise Rayleigh waves or bulk acoustic waves such as flexural, plate (e.g., Lamb) or thickness mode waves. The controller may be configured to control frequency or amplitude of the acoustic waves to control depth or rate of delivery of the therapeutic agent. The acoustic wave frequency may be, for example, in a range of 1 MHz to 10 GHz, a range of 1 MHz to 100 GHz, more than 10⁶ Hz, more than 10⁷ Hz, more than 10⁸ Hz, more than 10⁹ Hz, more than 10¹⁰ Hz, or more than 10¹¹ Hz. The primary acoustic excitation frequency may be, for example, between 10⁶ Hz and 10⁷ Hz, between 10⁶ Hz and 10⁸ Hz, between 10⁶ Hz and 10⁹ Hz, between 10⁶ Hz and 10¹⁰ Hz, between 10⁷ Hz and 10⁸ Hz, between 10⁷ Hz and 10⁹ Hz, between 10⁷ Hz and 10¹⁰ Hz, between 10⁸ Hz and 10⁹ Hz, between 10⁸ Hz and 10¹⁰ Hz, or between 10⁹ Hz and 10¹⁰ Hz. The acoustic wave amplitude may be, for example, in a range of 0.001 to 100 nm, more than 10³ nm, more than 10⁴ nm, more than 10³ nm, more than 10⁴ nm, more than 10⁵ nm, between 10² nm and 10⁵ nm, between 10³ nm and 10⁵ nm, between 10⁴ nm and 10⁵ nm, or between 0.001 to 10⁵ nm. The delivery depth may be in a range of 10 μm to 5 mm, for example, the depth of any of epithelial, dermal, intradermal, subdermal, mucosal epithelial, intramucosal, and submucosal tissue.

The device 10 in addition to generating a SAW or BAW frequency may further comprise an acoustic frequency generator (not shown) to simultaneously, either continuously or intermittently, generate another acoustic frequency signal to modulate the SAW frequency. The controller may further comprise a kilohertz range acoustic frequency generator. The kilohertz range acoustic frequency signal may have a frequency in a range of 1 Hz to 100 kHz. While it is not intended to be bound to any particular theory, it is believed that the modulation of the device SAW or BAW frequency by a kilohertz range acoustic frequency signal may enhance, permit or otherwise facilitate the megahertz or higher range acoustic frequency signal mediated delivery of the agent to certain depths of tissue.

The epithelial membrane 18 may form part of a subject's mouth, rectum or other parts of the gastro-intestinal system, genito-urinary and reproductive system including the vagina and uterus, respiratory system, skin, conjunctiva, eye and ocular system and the ear and auditory system. The subject may be a human or an animal.

In some embodiments of the present invention, the agent carrier of the device may comprise a number or network of micro channels surrounded by rigid walls for retention and/or delivery of various agents. The micro channels may be in fluid communication with a reservoir of the agent and extend partially or wholly through the device to its agent transfer surface. The micro channels may extend from within the interior of the agent carrier to the agent transfer surface of the agent carrier. The micro channels may, for example, be in the range of approximately 25 μm to 100 μm across when measured transverse to the direction of delivery. Additionally or alternatively, the micro channels may have a length of between approximately 0.5 mm to 2 mm. Any suitable cross-sectional and/or longitudinal geometry can be employed (e.g. cylindrical, conical etc.). The micro channels may terminate as pores at the agent transfer surface. During use of the device the agent may travel through the micro channels where it egresses through the pores of the agent transfer surface and into the tissue with which the agent transfer surface is in contact. A wide variety of shapes and sizes of pores may be utilised. The pores may, for example, be in the order of 10 μm to 100 μm in width, but may reach sizes of up to 1000 The micro channels may extend from the pores in the agent transfer surface at least partially or fully through the agent carrier body.

Devices according to the present invention may comprise a unitary agent carrier. Alternatively, the agent carrier may be formed from a plurality of layers assembled together in a stacked fashion. The stack of layers may comprise an agent transfer surface layer, and at least one other layer. The agent transfer surface layer may have holes extending through it to define at least a portion of micro channels in the body. In some embodiments a plurality of the layers have holes formed therein to enable agent to be transported from one layer to the next. Holes formed in one layer of the plurality of layers may be partially or completely aligned with holes in an adjacent layer so that a plurality of holes in a plurality of layers cooperate to form the micro channels. In some embodiments the holes decrease in diameter and increase in number from the first layer to the tissue-containing layer. The micro channels may have a varying cross-section along their length.

In some embodiments one or more reservoirs for storing the agent is partially or completely formed in the agent carrier of the device. The agent reservoir/s may comprise a void formed within the agent carrier body. Additionally or alternatively, the agent reservoir/s may be separate component/s in fluid communication with the agent carrier body. In some embodiments, the agent reservoir/s may be in fluid communication with one or more protrusions and/or pores existing in the agent transfer surface. Additionally or alternatively, the reservoirs may be in fluid communication with one or more of the micro channels. The micro channels may extend partially or wholly through the device.

The protrusions may define a portion or all of the agent transfer surface of the agent carrier. During use of the device, the protrusions may contact the target tissue and the agent to be delivered may surround them. The protrusions may extend outward from an inside of a void and terminate at the agent transfer surface. The void may be formed by a peripheral structure (e.g. a wall) and at least part of said peripheral structure may terminate at the agent transfer surface.

In some embodiments the peripheral structure terminates in a common plane with the protrusions. In other embodiments at least some of the protrusions defining the agent transfer surface extend outward from the void beyond the peripheral structure. In some embodiments, the protrusions may terminate in a plane and the peripheral structure may terminate short of the plane such that the protrusions extend beyond the peripheral structure.

The micro channels and/or agent reservoir/s and/or protrusions are generally defined by internal exposed surfaces within the agent carrier body. The internal exposed surfaces may be configured to possess predetermined hydrophilic, hydrophobic, and/or electro-conductive properties.

In some embodiments the protrusions are not microneedles and/or do not function as microneedles. Accordingly, the protrusions may not be intended to penetrate any layer of tissue to which the agent transfer surface is applied. In such embodiments, the function of protrusions includes engaging the target tissue by applying pressure resulting in a frictional force on the surface.

For example, the protrusions may not have a needle-like tip. Accordingly, they may not narrow to a point and their width may not decrease to near zero at the tip. The cross-section may be relatively constant, at least near the tip of the protrusions, and in some embodiments along their whole length. By way of non-limiting example, the width of the protrusions may not narrow by more than 20%, and in some embodiments less than 10% towards its tip. The protrusions may typically have a tip width greater than 10 μm. Thus the scale of the protrusions may also differ generally from that of microneedles. The protrusions may be constructed and or arranged such that they do not penetrate or otherwise enter an intact epithelial layer of the target tissue during normal/standard use of the device. The protrusions may aid in stabilising the device by the frictional force they apply when the device is placed in contact with the tissue. This may be particularly advantageous on mucous membranes that tend to have a low friction surface due to local mucous secretions. The protrusions may have a height to width aspect ratio (across their shortest cross sectional width) of between 1:1 to 10:1.

In various embodiments the protrusions may occupy more than 5% of the volume surrounding them in which agent is carried. The percentage is typically high enough so that the capillary force or other forces retain the agent within the agent carrier body against gravity or other forces caused by normal handling. In embodiments used with water-like agents, the device will typically have a density of projections of greater than 5% and most preferably greater than 10%.

### Delivery of Agents Using the Devices

Embodiments of the present invention provide methods and related devices that are useful for SAW mediated targeted drug delivery. Advantageously, embodiments of the SAW devices of the invention may be used in methods of treatment of diseases or disorders, or used in methods of immunisation to elicit or stimulate immune responses.

Embodiments of the present invention involve subjecting an agent to an acoustic excitation to controllably deliver an agent to a preferred depth range in tissues. The agent can be a fluid or carried in a fluid medium, e.g. by being dissolved, suspended or dispersed in a fluid medium, such as water, oil, an emulsion, a gel or the like. The agent can also be in a solid form such as a powder. The agent can be housed within, and delivered from, a variety of materials.

In certain embodiments, the acoustic excitation may enhance penetration of the agent into the tissue by among other things, increasing the rate or depth (or both) of movement of an agent into tissue that would otherwise without the acoustic excitation, diffuse into tissue at a slower rate or to a lesser depth (or both). The acoustic excitation may alternatively permit or enable penetration of the agent into the tissue by among other things, enabling the movement of an agent into tissue that would otherwise, without the acoustic excitation, not be able to move into tissue at all or would diffuse in amounts less than that required to obtain the desired effect.

Embodiments of the present invention utilises among other things, drug-containing devices utilising acoustic wave devices comprising a piezoelectric material to produce a surface (SAW) and/or bulk (BAW) acoustic wave by utilising one or more acoustic frequencies, that are applied directly to target tissue for the purpose of delivering drugs primarily to specific groups of target cells located at specific depths in or near target tissue. The energy imparted to molecules or particles contained in such devices by acoustic waves alone or acoustic waves modulated by other frequencies facilitates their delivery to the target tissue cells that lie in, or immediately below, the epithelial surface.

Direct apposition of the drug containing surface of the device to mucosal tissues, serves to mechanically minimise contact with mucous and enzymes that are resident on the surface of such tissue and retard the inflow of mucous and enzymes from surrounding areas. This ensures that the dose is delivered accurately and minimises the problems associated with local mucous drug clearance and local enzymatic degradation. It therefore solves one or more of the problems encountered and associated with intranasal and pulmonary mucosal drug delivery by vapors and sprays.

The agent to be delivered can include one or more molecules or particles or one or more molecules and particles in any combination. To give but a few examples, the agent can include chemically synthesised substances, biologics like proteins, amino acids, peptides, polypeptides, vaccines, nucleic acids, monoclonal and polyclonal antibodies, as well as nanoparticles or molecular machines. In preferred embodiments the agent is a pharmaceutical or pharmaceutical composition. The pharmaceutical or one or more active pharmaceutical components of a pharmaceutical composition may be, without limit, any one of: a synthesised compound, a naturally occurring compound, or a biopharmaceutical. The purpose of the delivery of the pharmaceutical or pharmaceutical composition to the biological tissues can be for any desired clinical reason including: treating, curing or mitigating a disease, condition, or disorder; attenuating, ameliorating, or eliminating one or more symptoms of a particular disease, condition, or disorder; preventing or delaying the onset of one or more of a disease, condition, or disorder or a symptom thereof; diagnosing a disease, condition, or disorder, or any agent intended to affect the structure or any function of the body. In other embodiments the agent can be an agent used for cosmetic purposes such as for cleansing, beautifying, promoting attractiveness, or altering the appearance of the body. The agent could also be a marker agent used for creating human or machine perceptible makings, e.g. ink or other. Other types of agents may also be used.

The acoustic excitation is the driving force for moving the agent through and/or from the device, and may enhance or enable the penetration of the agent from the device into tissue.

In preferred embodiments, the tissue can be any human or animal biological tissue, including mucous membranes, skin, nails and teeth. Preferably, the tissue is oral mucosa or ocular tissue. In other embodiments, the tissue is any plant tissue.

The acoustic excitation frequency may be in a range of 1 MHz to 100 GHz, more than 10⁶ Hz, more than 10⁷ Hz, more than 10⁸ Hz, more than 10⁹ Hz, more than 10¹⁰ Hz, or more than 10¹¹ Hz. The primary acoustic excitation frequency may be, for example, between 10⁶ Hz and 10⁷ Hz, between 10⁶ Hz and 10⁸ Hz, between 10⁶ Hz and 10⁹ Hz, between 10⁶ Hz and 10¹⁰ Hz, between 10⁷ Hz and 10⁸ Hz, between 10⁷ Hz and 10⁹ Hz, between 10⁷ Hz and 10¹⁰ Hz, between 10⁸ Hz and 10⁹ Hz, between 10⁸ Hz and 10¹⁰ Hz, or between 10⁹ Hz and 10¹⁰ Hz.

The delivery depth of the agent into tissue may be in a range of 10 μm to 5 mm.

The controlled delivery of the therapeutic agent across an epithelial membrane may elicit an immune response in a subject. The immune response induced in these aspects of the invention can be any one of a mucosal immune response, a systemic immune response, or both.

The acoustic excitation may comprise surface acoustic waves, bulk acoustic waves (e.g., flexural, plate (e.g., Lamb), or thickness mode waves), or combinations thereof.

To control depth or rate of delivery of the agent, the device may further comprise controlling operating parameters including (but not limited to) any one or more of the following:
application pressure;
acoustic frequency;
acoustic power level;
acoustic waveform;
acoustic application duration;
acoustic application duty cycle; and
acoustic direction.
the material that houses the drug and
the characteristics and ultrastructure of the agent transfer surface of the material

Preferably, the operational parameters are selected to deliver a chosen amount of agent to a selected depth within tissue. The person skilled in the art will appreciate that the optimal operational parameters needed to achieve a desired effect or response by application of agent to specific types of tissue can be determined by any combination of laboratory testing, other non-clinical means and by clinical investigations in animal models and human subjects.

Another way to control the depth or rate of delivery in the case of bulk transduction of the agent is for the device to include using a stack of one or more of each type of acoustic wave generating devices which serves to increase vibration amplitude and thus energy and power.

The method may involve delivering the agent to or beyond any one or more of the following tissues or tissue layers:
Mucous Membrane;
   Epithelium
   Sub-epithelium (lamina propria)
Mucosa;
   Sub-mucosa
   Mucous membrane vasculature
Cornea;
   Corneal epithelium
   Bowman's membrane
   Corneal stroma
   Descemet's membrane
   Corneal Endothelium
Conjunctiva;
Tenon's Fascia;
Episclera:
Sclera;
Choroid;
Choriocapillaris;
Bruch's membrane;
Retinal Pigment Epithelium;
Neural retina;
Retinal blood vessels;
Internal Limiting Membrane;
Vitreous;
Skin
   Epidermis
   Dermis
   Blood vessels
Teeth; and
Nails.

As can be seen, in each of the aspects and embodiments of the invention described herein, the target delivery site in a tissue may be defined as either being a particular layer or layers of a tissue, or alternatively be defined as a depth range. For example, the delivery of the agent may be defined in terms of being delivered to the Bowman's membrane of the cornea (ie a layer) or may be defined in terms of being delivered to a depth of approximately 5 to 15 μM (ie a depth range). The skilled person would be aware of what depth any given target layer is in any given tissue. The immune response induced in these aspects of the invention can be a mucosal immune response, a systemic immune response, or both. Preferably, at least a mucosal immune response is induced, and optionally a systemic immune response is also induced. It is considered that by selectively configuring the operational parameters of the agent applicator presently described, the amount of agent delivered to a selected depth or one or more layers of a tissue may be controlled.

For example, in some embodiments of the invention, there is provided delivery of the agent to induce at least a mucosal immune response by controlling the delivery of the agent such that the majority of the agent is delivered into the epithelial and sub-epithelial layer of the mucous membrane. Accordingly, in some embodiments of the invention, delivery of the agent induces at least a mucosal immune response. The agent may be applied using the operational parameters described herein, and preferably a sufficient dose of agent remains resident in the mucous membrane, at least temporarily, in order to induce an immune response in the mucous membrane. More specifically, a sufficient dose of agent remains resident at least temporarily in one or more of the epithelial or sub-epithelial layers of the mucous membrane.

The tissue may contain or comprise of an epithelial membrane which may be a mucosal membrane or a cutaneous membrane. For example, the mucous membrane may form part of a subject's ocular conjunctiva, mouth, rectum or other parts of the gastro-intestinal system, genito-urinary and reproductive system including the vagina and uterus, the respiratory system including the nasal mucosa, larynx, pharynx, bronchi and lungs. The cutaneous membrane is skin. The tissue may also be the cornea, the tympanic membrane of the ear, teeth and nails.

The methods of embodiments of the invention described herein can also include one or more of the steps of:
loading the absorbent or adsorbent material or reservoir with agent;
providing the absorbent or adsorbent material or reservoir holding the agent;
bringing an agent transfer surface of the device into direct or indirect contact with said tissue; and
dispensing the agent from the device to the tissue surface, wherein the step of dispensing the agent preferably includes generating an acoustic signal to cause or facilitate transportation of the agent to the tissue-contacting surface.

By indirect contact it would be understood that a substance such as a gel may be interposed between the agent transfer surface of the device and the tissue in order to optimise transmission of the acoustic signal.

As would be understood by the skilled person, the delivery of agent to one selected layer may not be absolute. For example, the operational parameters of the device may be configured to deliver a sufficient amount of the agent and by ‘sufficient amount’ it would be understood to comprise an amount of riboflavin-5-phosphate-sodium in the anterior corneal stroma sufficient to, in the case of the treatment of keratoconus as an example, crosslink collagen using UV-A light. However some of the agent may also be delivered to Descemet's membrane. This small amount of ‘overflow’ is not contemplated to be delivery to both the corneal stroma and Descemet's membrane in accordance with the invention. Rather, if it is intended that a sufficient amount of agent be delivered to both the corneal stroma and Descemet's membrane the specific operational parameters of the agent applicator would need to be configured in order to specifically achieve delivery of a sufficient amount of the agent to all desired layers. Similarly, delivery of the agent through, for example, the corneal stroma and Descemet's membrane may result in some of the agent remaining in either or both of those layers; but for the purposes of the invention, a sufficient amount of agent will be delivered to the underlying tissue.

In some embodiments of the present and previous aspects of the invention, delivery of an agent induces immunity against infections.

Embodiments of the method may further comprise modulating the frequency of the acoustic excitation by another acoustic signal (e.g. a lower frequency acoustic signal). The modulating acoustic frequency signal may, for example, have a frequency in a range of 1 Hz to 100 kHz, more than 10⁶ Hz, more than 10⁷ Hz, more than 10⁸ Hz, more than 10⁹ Hz, more than 10¹⁰ Hz, more than 10¹¹ Hz, between 10⁶ Hz and 10⁷ Hz, between 10⁶ Hz and 10⁸ Hz, between 10⁶ Hz and 10⁹ Hz, between 10⁶ Hz and 10¹⁰ Hz, or between 10⁶ Hz and 10¹¹ Hz. This modulated acoustic frequency signal may assist in transporting the agent to a given depth of tissue that would not be possible or efficient by using a single constant frequency alone. The modulated acoustic frequency signal may assist in transporting the agent to a desired given depth of tissue. The modulated acoustic frequency signal may also assist in transporting the agent to a given depth of tissue in a timeframe that is less than would be required using a single constant frequency alone.

The agent may be stored by any combination of one or more of, being absorbed by a porous or fibrous material, adsorbed onto or into a porous or non-porous material or housed in a reservoir contained within the material or external to it. The absorbent material may, for example, comprise any kind of porous material including paper, film or a porous manufactured material made from piezoelectric materials, silicons, metals, ceramics or plastics. The reservoir may, for example, comprise a microfluidic reservoir or fluid micro channels formed in a substrate like silicon, metal (including lithium niobate), ceramic or plastic. In some embodiments the agent is not stored in an absorbent. In some embodiments the agent is not stored in paper.

The adsorbent material may be any surface on a porous or fibrous monolithic material or the chemically or physically functionalised surface of the porous or fibrous material or a porous manufactured material made from piezoelectric materials, silicons, metals, ceramics or plastics. The adsorbent may also be any compound in powder or granule form. The reservoir may, for example, comprise a microfluidic reservoir or fluid micro channels formed in a substrate like silicon, metal (including lithium niobate), ceramic or plastic.

By having a solid or dry particulate form of an agent stored by absorption or adsorption within the apparatus, it may not be necessary, for example, to refrigerate certain drugs like biologics and vaccines. This may obviate the problem of maintaining cold-chain logistics for transporting vaccines in developing countries.

The acoustic excitation may be generated on a piezoelectric substrate or ceramic using an electroacoustic transducer controlled by a controller.

The electroacoustic transducer may comprise interdigital transducers or an electrode layer or electrode layers.

Embodiments of the present invention also provides apparatus, comprising:
an electroacoustic transducer on a piezoelectric substrate and controlled by a controller;
a fluid that can be fluidically coupled to tissue, and can be acoustically coupled to the piezoelectric substrate;
wherein the fluid source comprises a fluid comprising a therapeutic agent; and
wherein the controller is configured to control the electroacoustic transducer to generate the acoustic excitation that couple with the fluid to controllably deliver the therapeutic agent from the fluid source across the tissue.

The fluid source may comprise any combination of one or more of an absorbent, adsorbent or a reservoir. The absorbent may or may not, for example, comprise paper. The reservoir may, for example, comprise a microfluidic reservoir or fluid microchannels formed in a silicon or piezoelectric substrate.

The apparatus may further comprise a fluid couplant interposed between the fluid source and the piezoelectric substrate.

The apparatus may further comprise a superstrate interposed between the fluid couplant and the fluid source.

The acoustic excitation may have a frequency corresponding to the resonant frequency of the piezoelectric substrate. The acoustic excitation frequency may be in a range of 1 MHz to 1 GHz.

The device may further comprise an acoustic frequency generator to generate an acoustic frequency signal to modulate the underlying acoustic excitation. The acoustic frequency signal may have a frequency in a range of 1 Hz to 100 kHz, more than 10⁶ Hz, more than 10⁷ Hz, more than 10⁸ Hz, more than 10⁹ Hz, more than 10¹⁰ Hz, more than 10¹¹ Hz, between 10⁶ Hz and 10⁷ Hz, between 10⁶ Hz and 10⁸ Hz, between 10⁶ Hz and 10⁹ Hz, between 10⁶ Hz and 10¹⁰ Hz, or between 10⁶ Hz and 10¹¹ Hz.

### Delivery of Agents to the Eye and Treatment of Eye Conditions/Diseases

In some embodiments of the invention, the device may be used to deliver agent into the eye of a subject. The subject may, for example, be a human subject, a mammalian subject, or any other animal to which the device may effectively applied for the non invasive delivery of an agent into the eye. Delivery of the agent into the eye may be facilitated by contacting the device (specifically the agent transfer surface of the device) with any one or more of the corneal epithelium, corneal limbus and/or the conjunctiva overlying the sclera. The device may be used to propagate acoustic waves facilitating delivery of the agent to the interior of the eye by transport of the agent through the device and delivery of the agent through the corneal epithelium, corneal limbus and/or the conjunctiva overlying the sclera. The primary acoustic excitation frequency and power on and/or in the piezoelectric substrate of the device may depend on the target depth of delivery and in preferred embodiments may exceed 1 MHz (e.g. more than 10⁶ Hz, more than 10⁷ Hz, more than 10⁸ Hz, more than 10⁹ Hz, more than 10¹⁰ Hz, or more than 10¹¹ Hz). Supplementary, alternative or otherwise additional acoustic excitation frequencies of any wave type (including square, sine sawtooth) capable of modulating the primary acoustic excitation on and/or in the piezoelectric substrate may also be used and, for example may be less than or equal to the primary acoustic excitation frequency.

In applications where the agent transfer surface of the device is applied to the corneal epithelium, the agent may be delivered through the epithelium and where after passing through the corneal endothelium, it can enter the aqueous humor in the anterior chamber. The agent may be circulated within the aqueous which circulates in the anterior chamber, through the pupil and around the lens into the posterior chamber. The agent in the posterior chamber aqueous may contact the vitreous humor and blood vessels of the ciliary body and uveal blood vessels in the pars plana and, from there, be distributed via the choroidal vasculature to the posterior segment of the eye.

In applications where the agent transfer surface of the device is applied to the corneal limbus or the conjunctiva overlying any part of the sclera, the agent may penetrate though the conjunctiva and sclera to the choroidal vasculature and be transported through it posteriorly via the choroid capillary network (the chorio-capillaris) to the retina that lies internal to it separated from the choriocapillaris by Bruch's Membrane and the Retinal Pigment Epithelium which is the principal barrier to the entry of agents to the retina.

It is noted that in the context of delivering therapeutically effective agents into the eye the devices and methods of the present invention provide advantages over conventional/known methods.

The corneal epithelium is the major barrier to the entry of drugs into the cornea and eye. In the case of smaller agents (e.g. 500 Dalton or less, soluble) some are capable of passive diffusion through the cornea and/or sclera in therapeutic amounts, where conventional non-invasive delivery methods use eye drops or wafers (which include polymers). The wafer is physically held between the surface of the eye and the internal surface of the eye lid in the superior or inferior “fornix” (a cul de sac formed between the eyelids and the eye whose surface is covered by conjunctiva) whereby the agent can slowly leech out the drug. Eye drops commonly need to be applied 4-5 times a day. There is a lack of compliance commonly associated with eye drops including after application not closing the eye gently or not closing the eye for the period as required which both result in a significant reduction in the amount of drugs delivered to the eye. Wafers require a medical professional to insert them and can cause discomfort and irritation to the eye and infections. Compliance with the use of wafers is very much less than compliance with eyedrops. These methods/devices rely of the production of tears and for the patient to blink each of which may significantly vary in the population. While some small molecule drugs through eye drops and wafers may reach the posterior segment tissue including retina, various clearance mechanisms in the eye and adsorption into tissue preceding the posterior segment result in the amount of drug delivered to this area being significantly lower than that delivered to the cornea and anterior segment. On this basis, only mild inflammatory or infectious diseases disease in the posterior segment are commonly treated (as possible) through eye drops or wafers. Severe infections and inflammation in the posterior segment require that these small molecules are delivered by intra-vitreal injection to achieve a concentration that is therapeutically effective in this area. Any acute vision threatening disease is not suitable for treatment with eyedrops or wafers. Large molecules, including immunoglobulins and immunoglobulin fragment molecules used to treat severe vision threatening diseases including Macular Degeneration, Diabetic Macular Edema and Retinal Vein Occlusions cannot be delivered by eyedrops and these conditions are treated currently by delivering drugs by intra-vitreal injections.

The devices and methods of the present invention partially or wholly alleviate some or all of these shortcomings.

In general, the amount of drug delivered to the cornea and/or sclera by the devices of the present invention is greater and more rapid than can be achieved by using eye drops or wafers inserted in the cul-de-sac. The devices of the present invention are also capable of delivering therapeutically significant amounts of drugs to the choroid and ultimately to the retina which cannot be achieved by drops or wafers inserted in the cul-de-sac.

The amount of drug delivered to the cornea and/or sclera by the devices of the present invention is also predictable as the devices are directly applied against the tissue and operated for a certain period, the amount of drug remaining in the device following treatment can be measured, and delivery of the drug is not reliant on patient compliance or a minimum production rate of tears or blink rate. The device overcomes the barrier effect of the corneal epithelium.

The devices of the present invention also need to be applied less frequently than eye drops and not continuously applied over an extended time period like wafers in the cul del sac. The devices of the present invention device may include software that can monitor usage and compliance.

In the case of larger agents (e.g. more than 500 Dalton and/or are insoluble) which are incapable of passive diffusion through the cornea and/or sclera in therapeutic effective amounts, to the best of the inventors' knowledge there is no conventional non-invasive delivery method or device currently available to deliver such agents into the eye. Conventional delivery of these drugs is through intraocular (into the vitreous cavity) injection. Other methods include surgically implantable slow release wafers into the interior of the eye.

The devices and methods of the present invention partially or wholly alleviate some or all of these shortcomings.

In addition to the advantage of being non-invasive, the amount of drug delivered into the eye that is required for treating a disease in the choroid or retina is less than the amount required by conventional methods as following initial delivery through the conjunctiva/sclera, the drug is transported through the blood supply of the eye predominantly to the target tissue site and as is not diverted in relevant amounts away from the eye through various clearance mechanisms or absorbed or adsorbed into surrounding tissues that do not require treatment. A reduction in the amount of drug required delivered to the eye is advantageous as it reduces any side effects or risks associated with the drug including when it is cleared into the systemic circulation. For example “Avastin” (Bevacizumab) which is used to treat the wet form of age related macular degeneration can cause stroke through the drug entering the systemic circulation. Additionally, it reduces the cost of both treatment and manufacture. Furthermore, conditions and diseases of the eye such as, for example, Wet Age Related Macular Degeneration, Diabetic Macular Edema (DME) and infectious and inflammatory diseases of the choroid create breaks in Bruch's Membrane and retinal pigment epithelium (RPE) which permits neo-vascular and leaky choroidal vessels to enter the retina causing local haemorrhage and subsequent scarring. The most effective therapeutic target tissue for therapeutic agents is the choroid since the natural blood flow may carry the agent to the region of the retina where its integrity has been breached by neo-vascular tissue originating from the choroid.

In some embodiments, the devices and methods of the present invention can be used to treat conditions/diseases of the eye in a subject by delivering a therapeutically effective amount of an agent to a tissue. The subject may be any one or more of an animal subject, a mammalian subject, or a human subject. The condition/disease may be any that benefits from the non-invasive delivery of a therapeutic amount of an agent to a target tissue/component within the eye.

The term “therapeutically effective amount” as used herein will be understood to mean an amount of a given agent or mixture of agents that when administered to a subject, will have the intended therapeutic effect. The intended or full therapeutic effect may occur by administration of one dose of the agent or agent mixture, or alternatively may occur after administration of a series of doses. Thus, a therapeutically effective amount may be administered in one or more doses/administrations. The precise therapeutically effective amount needed for a subject will depend upon factors including, for example, the subject's age, size, health, the nature, location, and extent of the condition/disease, and/or the therapeutics or combination of therapeutics selected for administration. The skilled worker can readily determine a therapeutically effective amount of a given agent by routine experimentation.

By way of non-limiting example, the devices and methods of the present invention can be used for the treatment of keratoconus and myopia.

Keratoconus is corneal condition where, due to laxity of the corneal stroma's collagenous infrastructure, the cornea gradually becomes an increasing conical shape that causes irregular astigmatism which when it progresses cannot be corrected by spectacles or soft contact lenses. Historic treatment has been using hard contact lenses and if these become unsuccessful, corneal transplant surgery must be performed in order to regain useful vision.

Corneal collagen cross-linking is currently being used as a treatment modality option to halt the progression of keratoconus by stiffening the collagen ultrastructure of the corneal stroma. Corneal collagen cross-linking (CXL) requires riboflavin-5 phosphate sodium to be within the corneal stroma. The barrier effect of the corneal epithelium retards the entry of riboflavin-5 phosphate sodium. The majority of current techniques surgically remove the corneal epithelium so as to enable the delivery of riboflavin-5 phosphate sodium to the stroma. Despite the barrier being removed, riboflavin-5 phosphate sodium containing drops must be applied every one or two minutes (usually for a period of 30 minutes) before the corneal stroma contains a sufficient concentration of riboflavin-5 phosphate sodium for the next stage of treatment being exposure to Ultraviolet Light-A can proceed. Following riboflavin-5 phosphate sodium absorption, the cornea is exposed to UV light (typically 365-370 μm) for a time period of 30 minutes to induce collagen crosslinking. After treatment, the cornea is at risk of infection because the epithelium has been removed and the resulting ulcer must heal by the epithelium growing back to cover the defect which takes several days. To limit the severe pain, a “bandage” soft contact lens is applied and antibiotic eyedrops are used at least 4 times a day until the ulcer is healed. The Ophthalmologist needs to review the patient to ensure that healing is complete and that no infection has developed.

The devices and methods of the present invention can be used to non-invasively deliver riboflavin-5 phosphate sodium (and/or substitutes known in the art such as Glutaraldehyde [GD], Grape seed extract [GSE], and/or Genipin [GE]) into the cornea without removing or significantly weakening the corneal epithelium. For example, the agent transfer surface of a device according to the present invention can be contacted with the corneal epithelium. Acoustic waves propagated on and/or in the piezoelectric substrate of the device can be used to transport riboflavin-5 phosphate sodium through the device and deliver it through the corneal epithelium into the corneal stroma to a target depth. UV exposure can then be ordinarily used to induce collagen crosslinking. The corneal epithelium is not damaged by this non-invasive delivery, and the application can thus be repeated as frequently as necessary to achieve the desired outcome without subjecting a patient to the discomfort and risks associated with removing the corneal epithelium. The riboflavin-5 phosphate sodium can be delivered to the stroma in 3 to 5 minutes which is 6 to 10 times faster that by the invasive conventional method currently used.

Current conventional treatments available for eliminating the dependence on spectacles and contact lenses for myopia (near sightedness) involve invasive excimer laser corneal surgery to effectively flatten the contour of the cornea by laser ablation of the stroma or removal of the lens and replacing it with a plastic intra-ocular lens.

Ortho-Keratology is non-invasive and requires that a patient wears a rigid contact lens (an Ortho-K lens) overnight which is removed on waking. The rigid lens flattens the cornea and temporarily reduces myopia so that the patient can function without a visual aid during the day. The effect of the flattening wears off during the ensuing hours and the rigid contact lens is inserted again the following evening before sleep. The Ortho-K hard contact lens can create corneal ulceration and sleep disturbance if it is uncomfortable. So as to retain the corneal shape created by wearing an Ortho-K hard contact lens for a long period, after removal of the Ortho-K lens, and after removal of the corneal epithelium, riboflavin-5 phosphate sodium/UV-A light collagen cross linking has been used by some investigators in an effort to retain the flattened corneal shape. It is known that collagen cross-linking continues for some hours after the UV-A light treatment phase is complete. It would be advantageous if the Ortho-K lens could be worn immediately after treatment but this cannot be done because there is an ulcer on the eye following the removal of the corneal epithelium. Due to the corneal epithelium being surgically removed or weakened to facilitate riboflavin-5-phosphate-sodium uptake, it is generally not possible to repeat the procedure for a number of months.

The devices and methods of the present invention can be used to non-invasively deliver riboflavin-5 phosphate sodium to the anterior corneal stroma without removal of the corneal epithelium. This can be used in a novel treatment of myopia that includes the following steps:
remove an Orth-K hard contact lens after wearing it overnight;
using the devices and methods of the present invention to non-invasively deliver riboflavin-5 phosphate sodium to the anterior corneal stroma without removal of the corneal epithelium;
performing the conventional UV-A light collagen cross linking procedure; and
immediately following the conventional UV-A light collagen cross linking procedure, reapplying the Orth-K hard contact lens on the cornea for at least two hours (as collagen cross linking continues for at least two hours following cessation of UV-A light exposure). This enables the corneal collagen cross-linking to continue to stiffen the cornea whilst it is being moulded to its ideal shape by the Ortho-K hard lens; and
following the above steps, normal use of the Orth-K hard contact lens can resume.
   The non-invasive nature of this treatment among other things enables the procedure to be repeated as often as clinically required to be effective.

In addition to the novel treatment for myopia above, the devices and methods of the present invention can be used to non-invasively deliver riboflavin-5 phosphate sodium to the anterior corneal stroma without removal of the corneal epithelium as a novel treatment of keratoconus that includes the following steps:
using the devices and methods of the present invention to non-invasively deliver riboflavin-5 phosphate sodium to the anterior corneal stroma without removal of the corneal epithelium; and.
performing the conventional UV-A light collagen cross linking procedure. This procedure can halt the progression or partly reverse keratoconus.
   The non-invasive nature of this treatment among other things enables the procedure to be repeated as often as clinically required to be effective.
   Some patients with keratoconus may benefit having an extra step as outlined above of immediately following the conventional UV-A light collagen cross linking procedure, applying an Orth-K hard contact lens on the cornea for at least two hours (as collagen cross linking continues for at least two hours following cessation of UV-A light exposure). This enables the corneal collagen cross-linking to continue to stiffen the cornea whilst it is being moulded to its ideal shape by the Ortho-K hard lens.

The devices and methods of the present invention can be used to non-invasively deliver agents (e.g. therapeutic agents in therapeutically-effective amounts) to the posterior segment of the eye.

For example, in some embodiments the devices and methods of the present invention may be used to deliver a therapeutically effective amount of the agent for treating a condition or disease upon delivery to the posterior segment of the eye by contacting the tissue transfer surface of the device with the corneal epithelium. In this manner, the agent may be delivered into and through the corneal epithelium, Bowman's membrane, corneal stroma and corneal endothelium into aqueous humor. The agent may then circulate within the aqueous humor through the pupil and around the lens into the posterior chamber where it may contact one or more of the vitreous humor, ciliary body blood vessels, uveal blood vessels in the pars plana, and be distributed via the choroidal vasculature to the posterior segment of the eye.

In other embodiments, the devices and methods of the present invention may be used to deliver a therapeutically effective amount of the agent for treating a condition or disease upon delivery to the posterior segment of the eye by contacting the tissue transfer surface of the device with the conjunctiva overlying the sclera. In this manner, the agent may be delivered into and through the conjunctiva overlying the sclera, and the sclera, and then enter the uveal tract of the eye where it can be distributed via the choroidal vasculature to the choroid and retina in the posterior segment of the eye.

Accordingly and again by way of non-limiting example the devices and methods of the present invention can be used for the treatment of conditions/diseases localised in or emanating from the posterior segment of the eye. Acoustic waves propagated on and/or in the piezoelectric substrate of the device can be used to transport drug through the device and through the conjunctiva overlying the sclera and sclera to the choroidal vasculature and be transported through it posteriorly via the choroid capillary network (the chorio-capillaris) to the retina. The devices and methods can be used to non-invasively deliver therapeutically effective amounts of an agent to a target tissue in the posterior segment of the eye (e.g. sclera, fovea, anterior hyaloid membrane, vitreous humor, retina, choroid, optic nerve, optci disc). Non-limiting examples of applicable conditions/diseases include Age Related Macular Degeneration, Diabetic Macular Edema (DME), infectious disease, and inflammatory diseases. Others include inherited diseases of the retina which may potentially be treated by the introduction of RNA and its sub types, DNA, and other biologics to such tissue.

For the purpose of this specification, the word “comprising” means “including but not limited to”, and the word “comprises” has a corresponding meaning. The terms “include”, “for example”, “non-limiting example”, “comprises” and “comprising” will each be understood to be non-exhaustive in relation to the subject matter following them.

The above embodiments have been described by way of example only and modifications are possible within the scope of the embodiments that follow.

The invention will now be described in more detail, by way of illustration only, with respect to the following example. The example is intended to serve to illustrate this invention, and should not be construed as limiting the generality of the disclosure of the description throughout this specification.

### Example One

The SAW device 10 illustrated in FIG. 1 was used to controllably deliver albumin-fluorescein isothiocyanate (FITC-albumin) across epithelial membranes of pig lips. Fresh lips tissues from pigs were obtained from a slaughterhouse. The experiments were conducted within 2 hours after the animals were sacrificed. The porcine lips were exposed to FITC-albumin molecules with and without the acoustic waves. The exposure time was 5 s for both control and SAW experiments. The power or voltage was maintained at 0 mV (no voltage) for control, and approximately 4V for SAW experiments. The concentration and the amount of FITC-Albumin (30 μg/ml) used was maintained constant throughout the penetration depth study.

The fresh tissues were further snap frozen in isopentane chilled by liquid nitrogen with the aid of optimal cutting temperature compound (OCT) as the embedding medium. Samples were further wrapped in aluminium foil and stored at −80° C. until sectioned by a dermatome. The tissues were sectioned by a dermatome in a cryostat to create samples of contiguous layers of tissue of 50 μm thickness.

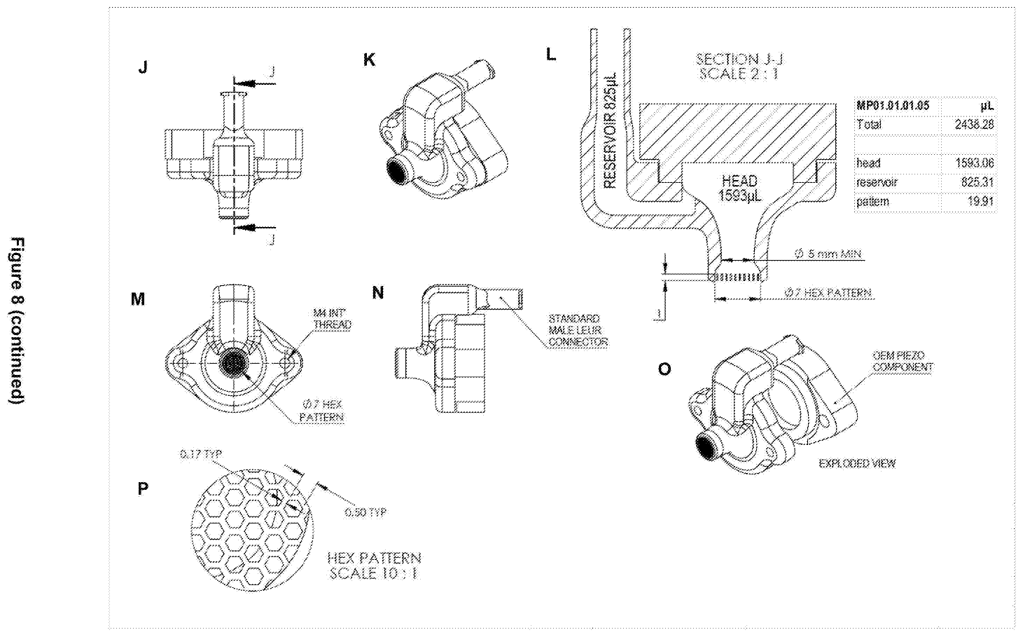

Fluorescent images of the samples were obtained and quantified using Image J software to calculate the normalised pixel intensity of the green channel. FIG. 7 and Table 1 below show the penetration depth of FITC-albumin molecules for the control and the SAW device 10.

TABLE 1

| Layer thickness | Control samples pixel | SAW samples pixel |
|---|---|---|
| (μm) | intensity | intensity |
| 100 | 7.52 | 37.192 |
| 150 | 7.421 | 19.733 |
| 200 | 6.617 | 19.835 |
| 250 | 6.126 | 14.041 |
| 300 | 7.488 | 11.229 |
| 350 | 7.405 | 16.047 |
| 400 | 7.589 | 12.274 |
| 450 | 6.886 | 9.935 |
| 500 | 7.715 | 9.958 |

The results show average pixel intensity for the control samples were consistently lower than those of comparable depth layers of tissue that was actuated with SAW by the SAW device 10. In the control, the FITC-albumin molecules diffused evenly and uniformly through the layers of the pig lips. In contrast, the SAW device 10 controllably delivered the FITC-albumin molecules across the epithelial layers of the pig lips to a depth of 500 μm. Tissue deeper than 500 μm was not examined.

### Example Two

In this study experiments were performed to determine the uptake of Riboflavin-5-phosphate-sodium, vitamin B2, a vitamin found in food and used for corneal collagen crosslinking (CXL) for the treatment of keratoconus. Keratoconus is a disorder of the cornea of the eye caused by a weakening of its collagenous ultrastructure which results in the progressive thinning and sagging of the cornea leading to visual dysfunction including blurry vision, near sightedness, irregular astigmatism and light sensitivity. The visual disturbance caused by the disorder cannot be improved by the use of spectacles. Treatment of keratoconus includes hard contact lenses, CXL and in cases where the cornea has been significantly compromised, corneal transplantation.

CXL treatment has been shown to strengthen corneal structures biomechanically and biochemically. Conventional CXL treatment involves the removal of the corneal epithelium, soaking the cornea with Riboflavin-5-phosphate-sodium solution for 30 minutes and then irradiating the cornea with ultraviolet light (UVA). The corneal epithelial layer is the main barrier to entry of drugs into the cornea (including Riboflavin-5-phosphate-sodium) and is removed to facilitate relevant amounts of Riboflavin-5-phosphate-sodium to enter into corneal stroma (substantia propria). Removal of the corneal epithelial layer (clinically known as “Epi-off” in CXL procedures) causes post-operative discomfort lasting for several days and can result in significant risks including irregular healing and infection.

Non-invasive delivery of Riboflavin-5-phosphate-sodium to the cornea is highly desirable as it overcomes the discomfort and risks associated with removal of the corneal epithelial layer. Unlike Epi-off procedures, non-invasive delivery of Riboflavin-5-phosphate-sodium to the cornea can be repeated without risk. The non-invasive delivery of Riboflavin-5-phosphate-sodium into the stroma in combination with CXL could become a future treatment option for myopia (short-sightedness).

### Aim

The purpose of this study was to determine if the inventors' handheld form beta (HFB) device was able to successfully delivery Riboflavin-5-phosphate-sodium non-invasively into all layers of the rat cornea without removal of the epithelial layer.

### Experimental Design

All experiments were conducted in accordance with the ARVO Statement for Use of Animals in Ophthalmic and Vision Research and with approval from the ANU Animal Experimentation Ethics Committee (Ethics ID: A2014/56). Adult Wistar rats were utilised in all experiments, which were born and reared under normal lighting conditions (˜5 lux light) and aged between 90 and 120 post-natal days at the time of use. Wistar rats were anaesthetised with an intraperitoneal injection of ketamine (100 mg/kg; Troy Laboratories, NSW, Australia) and Xylazil (12 mg/kg; Troy Laboratories). Once anaesthetised, Systane eye drops (Alcon, TX, USA) were applied every 2 minutes to maintain corneal lubrication. Animals were placed on a heat blanket to maintain body temperature at 37° C. and a cotton eye loop was applied around the eye to expose the anterior surface.

The inventors' HFB device (with A.A Lab Systems A-301 HS HV amplifier and RIGOL DG4062 Function/Arbitrary Waveform Generator) was loaded with 0.2% Riboflavin-5-phosphate-sodium (Riboflavin-5-phosphate-sodium-5-phosphate sodium #30-1598-25GM; PCCA, TX, USA), made up in sterile saline (0.9% NaCl), and applied to the centre of the cornea for 5 minutes. The settings on the device were as follows: transducer—1.6 MHz; amplifier—DC offset=off position; waveform generator—60 KHz sine waveform. The device was run for 5 minutes at 0, 3, 11 or 13 voltage peak to peak (VPP). The device contained an electrode to propagate acoustic waves on the lithium niobate substrate of the device, however the device did not contain nor was it connected to an external second electrode placed on or near the subject during operation of the device. Therefore, an electric circuit between the device and the eye tissue was not created.

In all animals, the left eye was treated as control with the device applied to the eye for 5 minutes but without any power applied to the device, while the right eye was the treated eye with the device active. Following delivery, the residual Riboflavin-5-phosphate-sodium was washed from the eye with normal saline. The animal was euthanized, the eyes removed and the eyes fixed in 4% paraformaldehyde for 5 minutes before being cryopreserved and immediately sectioned at 16 μm thickness.

Sections were visualised under a Nikon A1 confocal microscope using the green 488 channel and imaged using the 10× objective. Data analysis for quantification was performed with an n=3 with 8 regions per section, with the gain of the laser set to optimal fluorescence for the brightest section (gain setting 110) and maintained for all imaging.

Experimental Findings

In this report, “Riboflavin-5-phosphate-sodium” is used as an abbreviation for Riboflavin-5-phosphate-sodium-5-phosphate sodium as specified above.

### Delivery in the Cornea

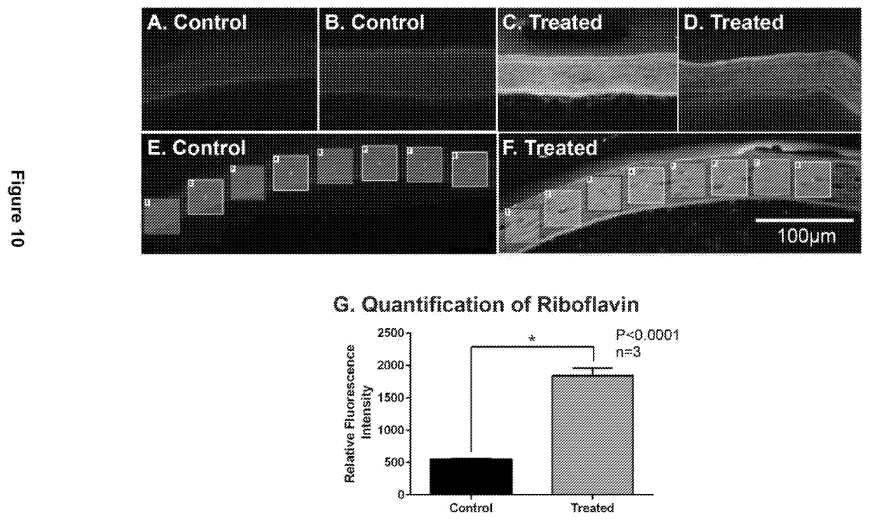

Without the application of voltage on the inventors' HFB device Riboflavin-5-phosphate-sodium fluorescence could not be detected in any structures of the eye including the cornea (FIG. 9A). Increasing the voltage from 3, 11 and 13 VPP showed an increase in Riboflavin-5-phosphate-sodium which initially showed small staining in the corneal epithelium (FIG. 9B), followed by labelling in all layers of the cornea at 11 and 13 VPP (FIG. 9C, 9D). Based on these preliminary results, 13 VPP was determined as the optimal parameter for determining penetration into the cornea and was used for the remainder of the data generated in this report.

### Quantification of Corneal Riboflavin-5-Phosphate-Sodium Delivery

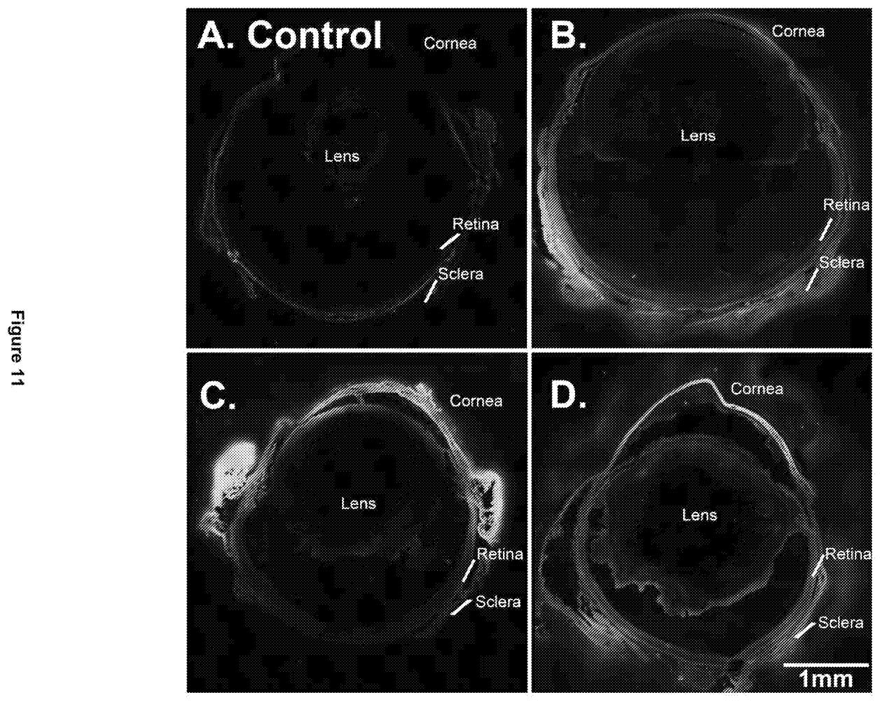

There was a clear and statistically significant difference (P<0.0001) between animals exposed to the device without voltage applied (FIG. 10A, 10B) compared to with the voltage applied (FIG. 10C, 10D). Quantification of this region was performed using average relative fluorescence intensity (RFI) for 8 regions on each corneal section as indicated in FIG. 10E, 10F. This quantification clearly showed a statistically significant difference (P<0.0001) between treated and control cornea with a nearly 4 times increase in RFI (FIG. 10G).

### Variability of Delivery to Cornea and Posterior Eye

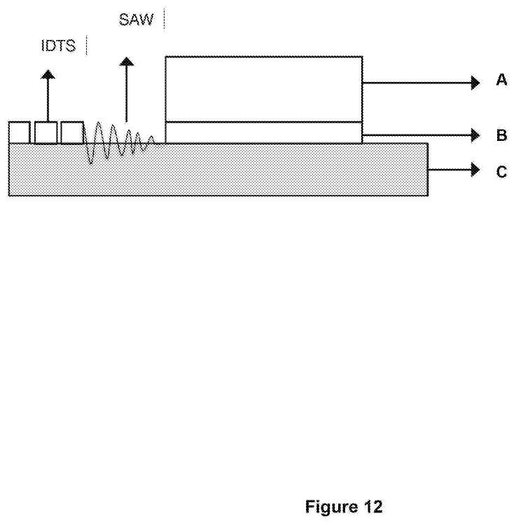

In all animals treated with Riboflavin-5-phosphate-sodium and an activated inventors' HFB device set at 13 VPP, we were able to detect labelling of Riboflavin-5-phosphate-sodium. The intensity of labelling and therefore delivery of Riboflavin-5-phosphate-sodium to the eye however was not consistent across all animals with some showing possible labelling in the sclera, limbus, lens and retina (FIG. 11). Whole eye sections of untreated controls (FIG. 11A) shows the background level of fluorescence, compared to 13 VPP treated animals (FIGS. 11B-11D). This small pilot study indicates that Riboflavin-5-phosphate-sodium was detectable mostly in the cornea in all animals, however it could also be visualised in the retina (FIG. 11B, lower left quadrant), limbus (FIG. 11C) and lens (FIG. 11D). The most likely explanation for this variability is differences in contact and pressure with the cornea and surrounding structures, such as the limbus, allowing for active transport of Riboflavin-5-phosphate-sodium throughout various structures of the eye. The penetration of Riboflavin-5-phosphate-sodium into other layers of the eye was an observation in this study, but not its main focus. Further detailed experimentation is required for confirmation and determining the mechanism by which the Riboflavin-5-phosphate-sodium was distributed to the posterior segment of the eye.

### Observations Regarding Posterior Segment Delivery

The anterior chamber and vitreous were dark and did not show any fluorescence. There are significant concentrations found in the posterior iris, ciliary body and in particular, the choroid. Given the dark aqueous and vitreous spaces, coupled with the highly fluorescent choroid, the distribution to the posterior segment mechanism must be via limbal, but mainly via pars plana choroidal vasculature. Further, given the rapid and very rich blood flow and anastomoses found in the Choroid, the Riboflavin-5-phosphate-sodium was distributed around the whole eye. Also, given that the choriocapillaris has fenestrations on the side of Bruch's membrane (which does not serve a barrier function), the abrupt change to a much lower fluorescence in the neural retina shows that the barrier function of the Retinal Pigment Epithelium remained intact. If the choroidal concentration was achieved via the vitreous, then the fluorescence in the neural retina would be much higher and (due to the barrier function of the RPE) the choroid would be less fluorescent than the neural retina.

### CONCLUSIONS

The inventors' HFB device was able to deliver Riboflavin-5-phosphate-sodium to all layers of the cornea at both 11 and 13 VPP, with the strongest visualisation at 13 VPP. Variability in the delivery was evident between animals and due most likely as a result of the subtle differences in the placement location of the device on the cornea and pressure applied by the device on the eye. This variability, although evident in the cornea, was most pronounced in the posterior eye where fluorescent Riboflavin-5-phosphate-sodium was visible in all layers of the retina, choroid and sclera in some animals. The study clearly showed that placing the device on the cornea resulted in the trans-epithelial non-invasive delivery of Riboflavin-5-phosphate-sodium to the cornea.

Further, by offsetting the placement of the device so that contact was made with both cornea and the adjacent conjunctival covered sclera overlying the Choroid in the region of the Pars Plana, Riboflavin-5-phosphate-sodium was non-invasively delivered to the posterior segment of the eye with Riboflavin-5-phosphate-sodium concentrated in the uveal tract. The latter has implications for the non-invasive delivery of anti-Vascular Endothelial Growth Factor (anti-VEGF) agents and anti-inflammatory drugs for the treatment of a variety of vision threatening conditions like Age Related Macular Degeneration, Diabetic Eye Disease and Posterior Choroiditis

### Example Three

### Introduction and Background

In this set of experiments, the effect of surface acoustic wave (SAW) at 30 MHz on FITC-Albumin and Fluorescein perfusion for different SAW powers and operating time was investigated.

### Materials and Procedure

Fluorescein sodium salt and FITC-Albumin were purchased from Sigma-Aldrich, Australia. Fresh porcine lips were obtained from diamond valley pork abattoir (Laverton, Melbourne) prior to the experiments. Surface acoustic wave (SAW) chip patterning of Chromium and Aluminum interdigital transducers (IDT) were fabricated using standard lithographic techniques at Micro Nano Research Facility (MNRF), RMIT University.

Fresh lips tissues from pigs were obtained from a slaughterhouse. The lips were wrapped in sterile gauze saturated in Krebs buffer during transportation to maintain the viability of the tissues. The experiments were conducted within 2 hours after the animals were sacrificed. The porcine lips were exposed to various molecules with and without the surface acoustic waves (SAW). The lips were washed immediately after the experiments to get rid of any reminent targeting molecules. The fresh tissues were further snap frozen in isopentane chilled by liquid nitrogen for further quantification studies.

Samples were wrapped in aluminium foil and stored at −80 degree until dermatomed. The tissues were dermatomed in a cryostat at a thickness of 50-micron layer until 1 mm for extensive penetration studies.

Tissue sectioning was performed at RMIT Bundoora and Melbourne University campuses. Fluorescent images of the samples were obtained using optical microscope (×10 lens), consistent light exposure of 600 ms. Acquired images were then quantified using ImageJ software to calculate the normalized pixel intensity of the green channel. These results were then calibrated against known samples, consequently converted into nanograms drug dose.

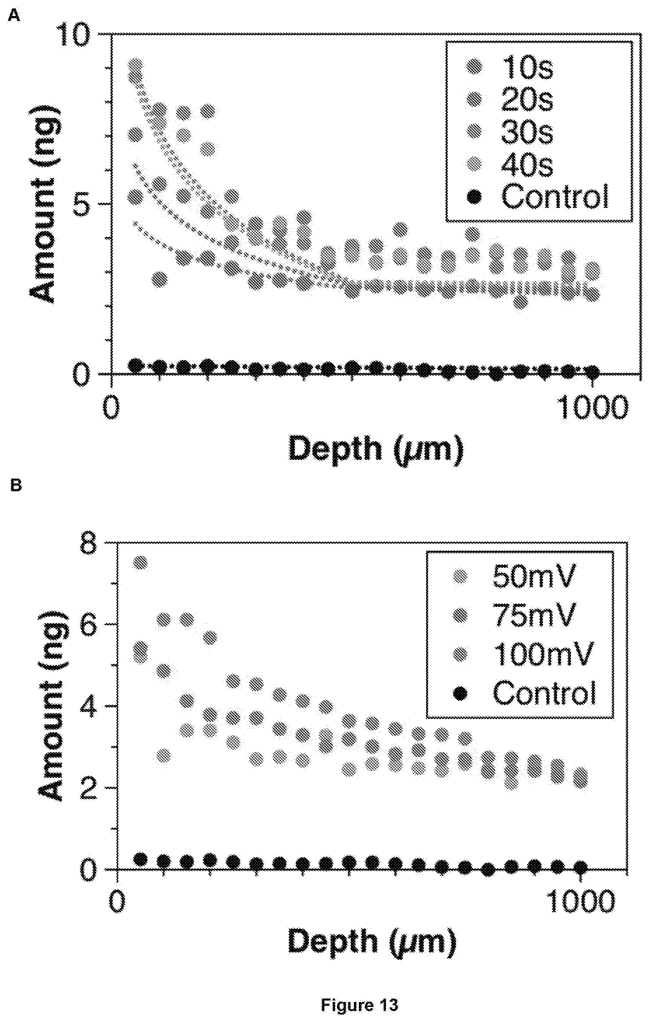

The model drug (FITC-albumin and fluorescein) used was placed directly on the device throughout the experiments. Fresh lips were approximately dissected into a 1 cm*1 cm cube and was placed on the drug. No coupling agent or loading substrate was used to transmit the vibrations from device into tissues. The inter-digital electrodes (IDTs) patterned on the lithium niobate (piezoelectric substrate) generated surface waves (SAW), which in turn drives the model drugs into the tissues. (FIG. 12)

### Results

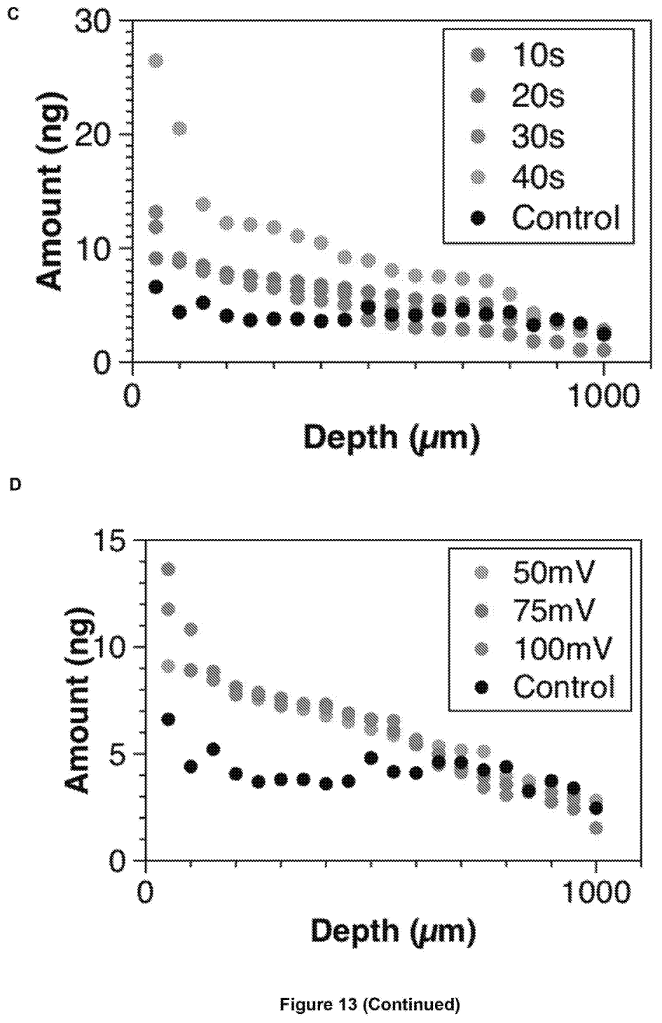

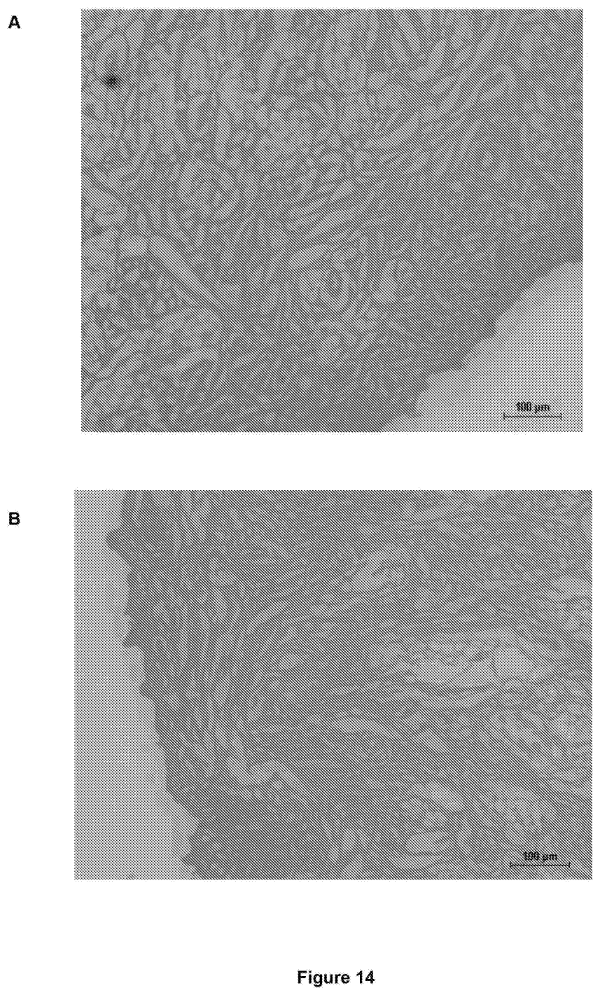

The results are summarised in FIGS. 13 and 14 and in Table 2 where SAW chip heating for different powers is also presented.

TABLE 2

| Power | 5 s (° C.) | 10 s (° C.) | 20 s (° C.) | 30 s (° C.) | 40 s (° C.) |
|---|---|---|---|---|---|
| 50 mV | 33 | 40.8 | 41.6 | 40.2 | 41.2 |
| 75 mV | 38.2 | 46.8 | 81.2 | 85.2 | 86.7 |
| 100 mV | 44.6 | 60.8 | 113.6 | 110.8 | 112 |

### Conclusion

SAW based perfusion of FITC-Albumin and Fluorescein was investigated under different exposure time and powers with the interdigital transducers and SAW device facing the lip. All experiments were conducted with 30 MHz frequency and the chip surface (including the IDTs) facing the lip. For Fluorescein, passive diffusion (control sample) was prevalent (FIGS. 13C and 13D) although SAW showed an improvement, especially for longer exposure time (˜40 seconds, FIG. 13C). For FITC-Albumin experiments, SAW perfusion showed an outstanding perfusion compared to passive diffusion (control sample). For passive control, due to the FITC-Albumin higher molecular weight, there was hardly any diffusion (FIGS. 13A and 13B).

1. A device, comprising:
an agent carrier comprising an agent transfer surface for delivery of an agent into a tissue, wherein the agent carrier comprises or is acoustically couplable to a piezoelectric substrate;
an electrode electrically couplable to the piezoelectric substrate; and
a controller electrically couplable to the electrode and configured to apply an electrical signal to the electrode to propagate an acoustic wave on and/or in the piezoelectric substrate which is capable of delivering the agent from the device into the tissue.

2. The device of claim 1, wherein the controller is configured to apply the electrical signal at a level which generates a primary acoustic excitation frequency on and/or in the piezoelectric substrate of more than 10⁶ Hz, between 10⁶ Hz and 10⁷ Hz, between 10⁶ Hz and 10⁸ Hz, between 10⁶ Hz and 10⁹ Hz, or between 10⁶ Hz and 10¹⁰ Hz.

3. The device of claim 1, further comprising an acoustic generator capable of generating a secondary acoustic excitation frequency capable of modulating a primary acoustic excitation frequency generated by the piezoelectric substrate, wherein the secondary acoustic excitation frequency is less than or equal to the primary acoustic excitation frequency.

4. The device of claim 1, wherein the acoustic wave is a surface acoustic wave (e.g. a Rayleigh surface acoustic wave).

5. The device of claim 1, wherein the device:
does not comprise an electrode for contacting the tissue surface, and/or
is not configured to utilise repulsive electromotive force to transport a charged agent into and/or through the tissue in contact with the agent transfer surface.

6. The device of claim 1, wherein:
the agent carrier comprises the piezoelectric substrate,
the piezoelectric substrate comprises the agent transfer surface, and
the agent is present on the agent transfer surface, and is optionally functionalised and/or lyophilised on the agent transfer surface.

7. The device of claim 1, wherein:
(i) the agent carrier comprises a multiplicity of micro channels extending partially or wholly through the agent carrier to the agent transfer surface enabling retention of the agent and/or transportation of the agent to the tissue; and
(ii) the micro channels extend from the interior of the agent carrier body and terminate as pores at the agent transfer surface, and/or
the agent transfer surface comprises a plurality of hollow micro protrusions in fluid communication with the micro channels; and
(iii) the micro protrusions are not microneedles and do not function as microneedles.

8. The device according to claim 1, comprising or in fluid communication with one or more reservoirs of the agent, wherein:
(i) the agent reservoirs comprise a void formed within the agent carrier body; and
(ii) the agent transfer surface comprises a plurality of protrusions in fluid communication with the agent reservoirs; and
(iii) optionally the plurality of protrusions extend outward from an inside of one or more of the voids and terminate at the agent transfer surface; and
(iv) the protrusions are not microneedles and do not function as microneedles.

9. The device of claim 8, wherein one or more of the voids is formed by a peripheral structure, and;
(i) the peripheral structure terminates in a common plane with the plurality of protrusions; or
(ii) the plurality of protrusions extend outward from the void beyond the peripheral structure; or
(iii) the plurality of protrusions terminate in a plane and the peripheral structure terminates short of the plane such that the plurality of protrusions extend beyond the peripheral structure.

10. A method for delivering an agent to an internal, layer within a target tissue, the method comprising:
contacting the target tissue with the agent transfer surface of the device of claim 1, and
applying an electrical signal to the electrode of the device to propagate an acoustic wave on and/or in the piezoelectric substrate of the device, and, thereby deliver the agent through the agent transfer surface to the internal layer of the target tissue.

11. The method of claim 10, wherein the method comprises delivering the agent into or through any one or more of: epithelium, sub-epithelium, mucosa, sub-mucosa, mucous membrane vasculature, nasal septum, cornea, conical epithelium, Bowman's membrane, corneal stroma, conical endothelium, conjunctiva, Tenon's fascia, episclera, sclera, choroid, choriocapillaris, Bruch's membrane, retinal pigment epithelium, neural retina, retinal blood vessels, internal limiting membrane, vitreous humour, teeth, a component of the gastro-intestinal system, a component of the genito-urinary, a component of the reproductive system (e.g. vagina, uterus), a component of the respiratory system, a component of the ocular system, a component of the auditory system, an eye, an ear, and a lip.

12. The method of claim 10, wherein:
the target tissue is intact tissue, and
the agent transfer surface is configured to inhibit or prevent mechanical penetration of a surface of the target tissue when in contact with it during standard use of the device.

13. The method of claim 10, wherein the target tissue is mucosal tissue, or the eye.

14. The method of claim 13, wherein the target mucosal tissue is intact, the agent transfer surface does not penetrate an intact epithelial layer of the target mucosal tissue during standard use of the device, and wherein delivery of a therapeutically effective amount of the agent into the target mucosal tissue induces mucosal immunity.

15. The method of claim 13, wherein the target tissue is the eye, and the method comprises contacting the agent transfer surface with corneal epitheliumand delivering a target amount of the agent into the cornea of the eye.

16. The method of claim 15, wherein:
the agent is delivered for the treatment of myopia or keratoconus,
the agent is a therapeutically effective amount of any one or more of Riboflavin-5-phosphate-sodium, Glutaraldehyde, Grape seed extract, and/or Genipin, and
the method farther comprises exposing the cornea to ultraviolet light following delivery of the therapeutic amount of the agent to the cornea for a time period sufficient to induce collagen crosslinking in the cornea.

17. The method of claim 16, further comprising repeating the delivery the therapeutically effective amount and the exposure to ultraviolet light within 1, 2, 3, 4, 5, 6, 7, 14, 21, 28, 42 or 60 days.

18. The method of claim 15, wherein:
the agent comprises a therapeutic amount of the agent for treating a condition or disease upon delivery to the posterior segment of the eye,
and the therapeutically effective amount of the agent
is delivered through the conical epithelium, Bowman's membrane, Conical stroma, Descemet's membrane and Corneal endothelium, into aqueous humor,
circulates within the aqueous humor through the pupil and around the lens into the posterior chamber,
contacts one or more of vitreous humor, ciliary body blood vessels, uveal blood vessels in the pars plana, and
is distributed via the choroidal vasculature to the posterior segment of the eye.

19. The method of claim 13, wherein:
the agent comprises a therapeutic amount of the agent for treating a condition or disease upon delivery to the posterior segment of the eye,
and the therapeutically effective amount of the agent
is delivered through the conjunctiva overlying the sclera, and the sclera,
enters the uveal tract of the eye,
is distributed via the choroidal vasculature to the choroid and retina in the posterior segment of the eye.

20. The method of claim 18, wherein the therapeutically effective amount of the agent comprises anti-Vascular Endothelial Growth Factor (anti-VEGF) agents, nucleic acids, and/or an anti-inflammatory drug, and is delivered for the treatment of Age Related Macular Degeneration, Diabetic Eye Disease, or Posterior Choroiditis.
